SEQUENCE LISTING (16 sequences)

SEQ ID NO: 1 - DNA, 23 bp, synthetic construct
  source 1..23: mol_type = other DNA
ccggactttgaaggcgtggattg

SEQ ID NO: 2 - DNA, 23 bp, synthetic construct
  source 1..23: mol_type = other DNA
cctcagcaaggactggttttcgg

SEQ ID NO: 3 - DNA, 22 bp, synthetic construct
  source 1..22: mol_type = other DNA
atcttagcggcacagagacgag

SEQ ID NO: 4 - DNA, 22 bp, synthetic construct
  source 1..22: mol_type = other DNA
tgtcatggtgcagctcctctgt

SEQ ID NO: 5 - DNA, 23 bp, synthetic construct
  source 1..23: mol_type = other DNA
ggagatgagcagtctgcctttcg

SEQ ID NO: 6 - DNA, 23 bp, synthetic construct
  source 1..23: mol_type = other DNA
atgagccaggtacaagagtgtgg

SEQ ID NO: 7 - DNA, 22 bp, synthetic construct
  source 1..22: mol_type = other DNA
aacagtgcaggagccgctttgt

SEQ ID NO: 8 - DNA, 22 bp, synthetic construct
  source 1..22: mol_type = other DNA
aggacgaaagccaggcagaaga

SEQ ID NO: 9 - DNA, 23 bp, synthetic construct
  source 1..23: mol_type = other DNA
ggaagacaaggtcaacagcctgt

SEQ ID NO: 10 - DNA, 21 bp, synthetic construct
  source 1..21: mol_type = other DNA
tccagtttccgctttgctcgc

SEQ ID NO: 11 - DNA, 23 bp, synthetic construct
  source 1..23: mol_type = other DNA
ggagttcacacgcctcaaagagg

SEQ ID NO: 12 - DNA, 22 bp, synthetic construct
  source 1..22: mol_type = other DNA
tcctcagcatctgccaggttgt

SEQ ID NO: 13 - DNA, 23 bp, synthetic construct
  source 1..23: mol_type = other DNA
tcttgaggttggctttctgccag

SEQ ID NO: 14 - DNA, 23 bp, synthetic construct
  source 1..23: mol_type = other DNA
ctgtgccagcaaagagaggagca

SEQ ID NO: 15 - DNA, 23 bp, synthetic construct
  source 1..23: mol_type = other DNA
agaagaggaagcaaaggaggctg

SEQ ID NO: 16 - DNA, 22 bp, synthetic construct
  source 1..22: mol_type = other DNA
aagtccactctctctccatcgg

(19) United States
(12) Patent Application Publication
(10) Pub. No.: US 2026/0337571 A1
(43) Pub. Date: Oct. 29, 2026

(54) Developmental cues from epicardial cells simultaneously promote cardiomyocyte proliferation and electrochemical maturation

(71) Applicant: Regents of the University of Minnesota, Minneapolis, MN (US)

(72) Inventors: Brenda Ogle, Minneapolis, MN (US); Andrew Khalil, Minneapolis, MN (US); Sophie Givens, Eugene, OR (US)

(21) Appl. No.: 19/660,772

(22) Filed: Apr. 28, 2026

Related U.S. Application Data
(60) Provisional application No. 63/795,839, filed on Apr. 28, 2025.

(51) Int. Cl. C12N 5/077; C12N 5/071

(52) U.S. Cl. CPC C12N 5/0657; C12N 5/0697; C12N 2501/15; C12N 2513/00

(57) ABSTRACT

Method, compositions and protocols for producing engineered heart tissue (EHT) by providing a population of human pluripotent stem cell-derived cardiomyocytes (hiPSC-CMs) and epicardial cells during a first culture period; and after the first culture period, introducing or generating within the culture one or more additional non-myocyte populations comprising epicardial-derived smooth muscle cells, endothelial cells, fibroblasts, or any combination thereof during a second culture period. The non-myocyte populations are provided in a temporally staged manner that mimics embryonic-to-fetal cardiac development, thereby promoting maturation, patterning, multicellular organization, or functional performance of the engineered cardiac tissue.

### CROSS REFERENCE TO RELATED APPLICATION

This application claims priority to and the benefit of provisional application Ser. No. 63/795,839, filed on Apr. 28, 2025, the contents of which are hereby incorporated in their entirety.

### STATEMENT

This invention was made with government support under HL160779 awarded by the National Institutes of Health. The government has certain rights in the invention.

### INCORPORATION OF SEQUENCE LISTING

The sequence listing that is contained in the file named “Sequence_Listing_U0110425US1.xml” which is 15,239 bytes in size as measured in Microsoft Windows operating system, was created on Jun. 29, 2026, and is filed electronically herewith and also incorporated herein by reference.

### BACKGROUND

The use of human induced pluripotent stem cell (hiPSC)-derived cardiac cells to study health and disease has exploded in the last decade with the advent of robust and reproducible methods for the differentiation of CM1. Unfortunately, CM generated from such protocols are largely immature. This is evident by their small and rounded morphology, sarcomere disorganization, automaticity, the primary use of glycolysis for adenosine triphosphate (ATP) production, the expression of fetal myofibril isoforms, a fetal ion channel composition and lack of expression of major calcium handling proteins of the sarcoplasmic reticulum and T-tubule. Copious efforts to drive hiPSC-CM maturation in vitro have been attempted over the last decade with various levels of success. Approaches to drive maturation include the use of 3D culture systems that more accurately mimic the complex milieu of signals CM received in vivo such as organoids, engineered heart tissues (EHTs), and 3D printed chambered structures (Lemonie, M. et al. 2017). The switch from 2D to 3D culture alone promotes some level of CM maturation (Correia, C et al 2018, Ergir, E et al. 2022, Branco, M et al. 2019). Further, in EHTs the imposition of increased mechanical “afterload” enhances force generation and maturation (Leonard, A et al. 2018). The use of electrical stimulation on EHTs, implemented to mimic development and “exercise” the CM, yields significant increases in CM electrical and mechanical maturation (Ronaldson-Bouchard, K (2018)). Additionally, some more user-friendly techniques exist including the use of “maturation media” to impose a switch from glucose to fatty acid-based metabolism and treatment with small molecule mitogenactivated protein kinase (MAPK) inhibitors that artificially imposes the downregulation of the MAPK pathway seen in human ventricular tissues during development (Feyen D. et al. 2020). Finally, the coculture of CM with other cell types present in the postnatal heart such as cardiac fibroblasts, endothelial cells, and epicardial-derived cells can enhance hiPSC-CM maturation (Beauchamp, P. et al. 2020, Giacomelli, E. et al. 2020).

Most of these methods impose changes characteristic of post-natal heart development. The stark changes in afterload and preload caused by birth that result in developmental cardiac hypertrophy, the electrical stimulation present from the development of the cardiac pacemaker system, the switch to fatty acid metabolism, and the inhibition of pathways such as MAPK do not occur until postnatal development. Freshly differentiated hiPSC-CM resemble early fetal CM that are largely undefined in patterning that takes place during embryonic development; during embryonic development atrial, ventricular, compact, and trabecular myocardium show distinct, spatial, genetic, and functional patterning.

### SUMMARY

Aspects of this disclosure relate to methods of producing engineered heart tissue (EHT), comprising providing a population of human pluripotent stem cell-derived cardiomyocytes (hiPSC-CMs); contacting the hiPSC-CMs with epicardial cells (ECs) during a first culture period; and after the first culture period, introducing or generating within the culture one or more additional non-myocyte populations comprising epicardial-derived smooth muscle cells, endothelial cells, fibroblasts, or any combination thereof during a second culture period, wherein the non-myocyte populations are provided in a temporally staged manner that mimics embryonic-to-fetal cardiac development, thereby promoting maturation, patterning, multicellular organization, or functional performance of the engineered cardiac tissue.

culturing the co-culture under conditions comprising transient inhibition of TGFβ signaling sufficient to maintain or expand an epicardial cell state during an initial stage; and subsequently reducing, removing, or discontinuing the inhibition of TGFβ signaling to permit lineage progression of the epicardial cells into one or more epicardial-derived cell types comprising fibroblasts, smooth muscle cells, or both, thereby mimicking an embryonic-to-fetal progression mimicry process in vitro.

The additional non-myocyte population comprises fibroblasts.

Endothelial cells and epicardial-derived smooth muscle cells are introduced during the second culture period before or after fibroblasts are introduced.

In one or more embodiments, the additional non-myocyte populations are introduced sequentially rather than simultaneously.

At least one of the additional non-myocyte populations is generated by directed differentiation of cells already present in the culture rather than by exogenous addition of fully differentiated cells.

In one or more embodiments, the fibroblasts are epicardial-derived fibroblasts that are generated in situ from epicardial cells.

In one or more embodiments, the epicardial-derived smooth muscle cells are generated in situ from epicardial cells or the endothelial cells are generated in situ from a vascular progenitor population present in the culture.

The timing of directed differentiation is controlled by changing one or more pathway-modulating agents at prescribed time points. The pathway-modulating agents comprise a TGFβ pathway inhibitor.

Another aspect of the present disclosure relates to an engineered heart tissue (EHT) composition, comprising cardiomyocytes derived from human induced pluripotent stem cells (hiPSCs); and one or more additional cell populations derived from hiPSCs selected from: epicardial cells, fibroblasts, endothelial cells, smooth muscle cells, and macrophages or myeloid lineage cells, wherein the cells are arranged within 2D layer or a 3D matrix to form EHT that mimics developmental multicellular interactions of human myocardium.

The cells are arranged in the 3D matrix and the 3D matrix comprises a biopolymer comprising fibrin.

In one or more embodiments, all cellular components of the EHT are derived from human induced pluripotent stem cells.

One or more of the cell populations comprise genetically encoded reporter constructs configured to report functional maturation or cellular state within the EHT.

One or more reporters selected from the group comprising reporters for structural cardiomyocyte maturation consisting of cTnT, a-actinin, and MYH7/MYH6; reporters for electrochemical maturation consisting of KCNJ2 and RYR2; reporters for electromechanical maturation consisting of GJA1 and NRAP, reporters for metabolic maturation consisting of PGCla and TOMM20; reporters for stress or injury markers consisting of NPPB and NPPA; reporters for cell death consisting of a caspase 3 activity sensor; reporters for fibroblast activation consisting of TE7/ACTA2; reporters for macrophage identity consisting of CD68, CD163, and MerTK, reporters for inflammatory signaling consisting of IL-6, TNFa, and IL-1b, and reporters for remodeling consisting of TGFb and IL-10.

Another aspect of the present disclosure relates to a method of producing an engineered heart tissue (EHT), comprising assembling human pluripotent stem cell-derived cardiomyocytes (hiPSC-CMs) with one or more precursor or differentiated non-myocyte populations in a two-dimensional culture, a three-dimensional culture, a hydrogel, a fibrin gel, an engineered heart tissue, or a bioprinted construct; and controlling the timing of appearance of epicardial cells before the appearance of fibroblasts and before or concurrent with the appearance of endothelial cells and smooth muscle cells, such that the EHT undergoes a staged developmental sequence that models embryonic-to-fetal cardiac multicellular interactions.

The method can further include producing one or more additional cardiac-supporting cell types selected from endothelial cells, epicardial-derived smooth muscle cells, and fibroblasts, by either: (i) introducing additional cardiac-supporting cell types into the culture at prescribed time points, or (ii) directing differentiation of cells present in the culture into said additional cardiac-supporting cell types at prescribed time points.

The later stage is initiated by withdrawal or reduction of TGFβ inhibition.

Fibroblasts are produced later than endothelial cells or smooth muscle cells.

### DESCRIPTION OF THE FIGURES AND TABLES

FIG. 1 (A-H). Differentiation and purity of hiPSC-EPC, EPD-FB, and CM. A) Schematic of the hiPSC-CM small molecule differentiation protocol. B) Brightfield image of D24 CM post replating and lactate purification as well as C) flow cytometry for CM before engineered heart tissue seeding using M4-hiSPCs as a negative control for cTnT gating. D) Schematic of the hiPSC-EPC small molecule differentiation protocol. E) A brightfield image of the hiPSC-EPCs and F) flow cytometry for the epicardial marker WT1 for M4-hiPSCs as a negative control and P2-4 EPCs from the M4, F7, and CCND2 hiPSC-lines used for this study. G) A schematic of the hiPSC-EPD-FB differentiation protocol and H) a brightfield image of the EPD-FBs. I) Flow cytometry for the fibroblast marker TE7 to assess the purity of the EPF-FB differentiation using M4-hiPSCs as a negative control for gating. Scale 50 μm.

FIG. 2. (A): representative images from E10 (left), E12 (middle), and E17 (right) mouse hearts stained for aActinin (green) for CM sarcomere visualization and DAPI (blue); (B): representative images of wheat germ glutenin (WGA—green) for cell membrane visualization, nkx2.5 (red) marking first heart field cardiomyocytes and DAPI for nuclei visualization (blue) from E10 (left), E12 (middle) and E17 (right) mouse heart sections; (C) representative images of mouse heart sections stained for nkx2.5 (red) marking first heart field cardiomyocytes, Ki67 (green) marking proliferation and DAPI (blue) marking the nucleus from E10 (left), E12 (middle) and E17 (right). (C): quantified average percent proliferating CM as determined by the co-localization for Ki67 with nkx2.5 and DAPI. Scale 100 μm. (D): quantified CM sarcomere length, FIG. 1E: CM area, FIG. 1F; CM perimeter and (G): the percentage of proliferating CM at each developmental timepoint. For (D-F) the violin plot center dashed line represents the median and outer dashed lines represent upper and lower quartiles of the distribution. For (G): the bar graph and error bars represent the mean±STDEV and each dot represents the average percent Ki67 and nxk2.5 copositive nuclei from multiple sections across E10 (n=8), E12 (n=8) and E17 (n=9) embryonic mouse hearts. (D): E10 (n=87), E12 (n=101) and E17 (n=108) sarcomeres across E10 (n=8), E12 (n=8) and E17 (n=9) embryonic mouse hearts were measured. For (E-F) E10 (n=116), E12 (n=122) and E17 (n=128) were measured. *p<0.05, **p<0.01, ***p<0.001, and ****p<0.0001.

FIG. 3 (A-B). EHT cross-sectional area measurements for twitch force calculation. A) A bar graph of the average cross-sectional area for each of the force-generating EHT conditions. B) A bright field image with the cross-sectional area of the EHTs highlighted, followed by phalloidin (green) and DAPI (blue) images for better cellular visualization within the EHT cross-sections. Scale 250 μm.

FIG. 4 (A-D). EHT phalloidin staining and OrientationJ coherency measurements. Representative transverse cross-sections of EHTs showing F-actin (green—phalloidin—top) and the orientation color map (bottom) showing the average angle of each F-actin fiber. Scale 250 μm for whole sections and 20 μm for zoomed-in fields of view.

FIG. 5. (A-J). Epicardial cells increase CM proliferation in 2D co-cultures. A) 2D co-culture experimental timeline where red text indicates experimental variables. Where either epicardial cells (EPCs) were seeded on day 27 or not, and each condition with or without EPCs was maintained in LASR media with or without TGF-Beta inhibitors (+/−SB). Cells fated for flow cytometry were incubated in EdU for 24 hours before fixation while the others were assessed functionally. B) Flow cytometry for cardiac troponin T (cTnT) indicating the percentage of CM in each experimental condition. C) Flow cytometry for cTnT and myosin light chain 2 (MLC2v) indicating the percentage of ventricular CM in each condition. D) Representative IHC images from M4-hiPSC 2D co-cultures at day 35 stained for cTnT (green), WT1 (red-epicardial cells) and the nucleus (DAPI-blue). E) Image quantification of WT1 area/DAPI area to determine the percentage of epicardial cells in all four conditions. F) Representative images of cTnT (green) and proliferating cells after 24-hours EdU incubation (red) and G) Flow cytometry quantification of cTnT+EdU+ and H) cTnT+Ki67+percentage for all experimental replicates. I) Representative M4 flow cytometry histograms of co-cultures with visualization of the cTnT+ population (CM count) with EdU or J) Ki67 on the x-axis. Scale 50 μm. Bar graphs represent the mean±STDEV. For (B), (E), (G), and (H) n=3 independent experiments for each hiPSC-line, (M4, F7, and CCND2) and (C) n=3 independent experiments for two of the hiPSC-lines (M4 and F7). *p<0.05, **p<0.01, ***p<0.001, and ****p<0.0001 for (C), (E), (G) and (H).

FIG. 6 (A-D). Flow cytometry for cardiomyocyte proliferation. A) Representative flow cytometry plots for each co-culture condition with cTnT on the x-axis and EdU on the y-axis as well as another set with B) cTnT on the x-axis and Ki67 on the y-axis. C) Quantification of flow cytometry for cardiomyocyte proliferation showing the percent cTnT+EdU+ population as well as D) the percent cTnT+Ki67+ population. (C-D) Depict bar graphs and error bars representing the mean±STDEV for n=3 independent experiments for each condition. *p<0.05, **p<0.01, ***p<0.001, and ****p<0.0001.

FIG. 7 (A-G). Co-culture composition. A) Brightfield images of all co-culture conditions at D35. Scale 50 μm. B) Quantification of WT1 Area/DAPI area indicating the percentage of epicardial cells in each co-culture condition broken down by line. C) Quantification of the percentage cTnT+ population from flow cytometry data broken down by lines. D) Representative flow cytometry data for cTnT. E) Quantification of the cTnT+MLC2v+ population for both lines quantified. F) Flow cytometry controls showing the cTnT+ and MLC2v on the x-axis with the day 24 CM stained for cTnT only (left) and D24 CM before treatment with ascorbic acid (right). G) Representative flow cytometry chart of the cTnT+MLC2v+ population for each co-culture condition at the experimental endpoint. (B-C) and (E) Depict bar graphs and error bars representing the mean±STDEV for n=3 independent experiments for each condition. *p<0.05, **p<0.01, ***p<0.001, and ****p<0.0001 for (B).

FIG. 8 (A-M). Epicardial cells enhance CM function in 2D co-cultures. A) Representative direct co-culture calcium transient (CaT) traces of CM from the M4-line for each condition. Quantification of CaT parameters B) time to peak, C) maximum amplitude, and D) CaT upstroke velocity. E) Representative patch clamp trace for the CM+EPC(+SB) and CM (+SB) conditions. Quantification of patch clamp parameters F) beating frequency (f), G) the AP duration at 90% repolarization (APD90), H) the AP maximum upstroke velocity (V0,max) and the maximum diastolic potential (MDP). J) The maximum and minimum traction stress maps for a representative CM determined using traction force microscopy (TFM). K) Axial and transverse force versus time plot for three contractions of a representative cell. Scale 20 μm. L) CM contractile force determined by TFM and M) CM contractile force normalized to ell area. (B-D) Box and whisker plots with +marking the mean. (B-D) Data compiled from n=3 independent experiments from each hiPSC-lines (M4, F7, and CCND2). (F-I) and (L-M) Center lines and error bars represent the mean±STDEV. For (F-G) and (I) CM+EPC(+SB) (n=11) and CM (+SB) (n=10) and for (H) CM+EPC(+SB) (n=8) and CM (+SB) (n=7) cells across n=3 independent experiments from the M4-line and (L-M) CM+EPC(+SB) (n=41), CM+EPC→DC (n=74), CM (n=60) and CM (+SB) (n=54) across n=3 independent experiment from each hiPSC-line. *p<0.05, **p<0.01, ***p<0.001, and ****p<0.0001.

FIG. 9 (A-C). 2D Calcium transients by hiPSC-line. Quantification of the calcium transient parameters A) time to peak, B) maximum amplitude, C) upstroke, and D) downstroke velocity under 1 Hz electrical stimulation broken down by hiPSCline. Each graph is a box and whisker plot where the (+) represents the mean and the data represents three fields of view per well from three wells per replicate and n=3 independent experiments per line. *p<0.05, **p <0.01, ***p<0.001, and ****p<0.0001.

FIG. 10 (A-C). 2D traction force microscopy by hiPSC-line. The traction force microscopy for A) M4-CM, B) F7-CM, and C) CCND2-CM showing the force (left) and the force normalized by cell spread area (right) for each co-culture condition. Each data point represents one cardiomyocyte. The center line and error bars represent the mean±STDEV across n=3 independent experiments. For (A) CM+EPC(+SB) (n=10), CM+EPC→DC (n=31), CM (n=28) and CM (+SB) (n=19). For (B) CM+EPC(+SB) (n=15), CM+EPC→DC (n=24), CM (n=16) and CM (+SB) (n=12) and for (C) CM+EPC(+SB) (n=16), CM+EPC→DC (n=19), CM (n=16) and CM (+SB) (n=23) *p<0.05, **p<0.01, ***p<0.001, and ****p<0.0001.

FIG. 11 (A-G). Epicardial cells modestly increase CM morphologic maturation. A) Representative IHC images of 2D co-cultures replated onto glass slides and stained with aActinin (green) for sarcomere visualization, MLC2v for ventricular phenotype validation (red), and DAPI (blue) for nuclear visualization. Scale 20 μm. B) Violin plots showing quantified sarcomere length C) CM area and D) the percent multinucleated CM. E) Representative transmission electron micrograph (TEM) images with red arrows indicating mitochondria in close apposition to sarcomeres and F) the quantified mitochondrial cross-sectional area (CSA). G) TEM image of sarcomeres showing the Z-line (Z), I-band (I), and A-band (A) for each condition. Scale 1 μm. In (B), (C), and (F) violin plots center dashed line represents the median, and outer dashed lines represent the upper and lower quartiles. In (B-C) the data represents CM across n=3 independent experiments for all three hiPSC-lines (M4, F7, and CCND2) for (B) CM+EPC(+SB) (n=784), CM+EPC→DC (n=839), CM (n=799) and CM (+SB) (n=713), and for (C) CM+EPC(+SB) (n=878), CM+EPC→DC (n=878), CM (n=873) and CM (+SB) (n=817). For (D) the bar represents the mean±STDEV for n=3 independent experiments for all three hiPSC-lines (M4, F7, and CCND2) and each dot represents the averages of 5 fields of view across three wells for each independent experimental replicate. In (F) the CSA of CM+EPC(+SB) (n=79) and CM (+SB) (n=81) mitochondria across 10 cells per condition for n=1 independent experiment from the M4-hiPSC line was measured. *p<0.05, ** p<0.01, ***p<0.001, and ****p<0.0001.

FIG. 12 (A-C). 2D co-culture cardiomyocyte morphology by line. The morphologic assessment of CM replated onto glass slides at D35 and fixed in diastole 24 hrs later showing quantification of CM sarcomere length (left), area (middle), and the percent multinucleated (right) for A) M4-CM, B) F7-CM and C) CCND2-CM. The sarcomere length and area violin plot center dashed line represents the median and outer dashed lines represent upper and lower quartiles. The multinucleated percent bar graph represented the mean±STDEV where each data point is the average percent of multinucleated CM across three wells and 5 fields of view per well for each independent experimental replicate with n=3 independent experimental replicates per condition. In (A) for sarcomere length CM+EPC(+SB) (n=276), CM+EPC→DC (n=286), CM (n=272) and CM (+SB) (n=224) and for CM area CM+EPC(+SB) (n=275), CM+EPC→DC (n=280), CM (n=270) and CM (+SB) (n=224). In (B) for sarcomere length CM+EPC(+SB) (n=202), CM+EPC→DC (n=265), CM (n=229) and CM (+SB) (n=181) and for CM area CM+EPC(+SB) (n=292), CM+EPC→DC (n=192), CM (n=263) and CM (+SB) (n=284). In (C) for sarcomere length CM+EPC(+SB) (n=410), CM+EPC→DC (n=393), CM (n=407) and CM (+SB) (n=409) and for CM area CM+EPC(+SB) (n=416), CM+EPC→DC (n=388), CM (n=503) and CM (+SB) (n=413). p<0.05, **p<0.01, ***p<0.001, and ****p<0.0001.

FIG. 13 (A-G). Single-cell RNAseq reveals unique vCM phenotype in epicardial and epicardial derived co-cultures. A) Single-cell RNAseq UMAP of clustering where 14 different clusters (0-13) were identified. B) UMAP of each condition where the clusters were labeled into 7 major cell identities: ventricular CM (vCM—Clusters 0, 3, 5, 9 and 10), proliferative vCM (Clusters 2, 6 and 7), epicardial cells (EPC—Cluster 1), proliferative EPCs (Cluster 8), epicardial to epicardial derived cells (EPC→DC-Clusters 4 and 11), atrial CM (aCM—Cluster 12) and non-differentiated cells (Cluster 13). C) Graph of the percentage of CM that fall into each of the 6 CM categories listed. D) Heat map of cardiac genes of interest for the bulk vCM clusters present in the CM-only controls (Cluster 0), CM+EPC(+SB) (Cluster 3), and CM+EPC→DC (Cluster 5). ORA for biological processes (BP) showing a dot plot for the E) upregulated and F) downregulated pathways for the three bulk vCM clusters as compared to all other vCM clusters. G) Heat map of some of the differentially expressed genes contributing to enrichment in four BP pathways 1) Cardiac Ventricle Development, 2) Regulation of Canonical WNT Signaling Pathway, 3) Cardiac Conduction, and 4) Response to Calcium Ions.

FIG. 14 (A-D). Single-cell RNAseq clustering of CM co-cultures. A) Graph showing the proportions of cells from each condition in each cluster. B) A heat map of the top 15 differentially expressed genes (DEG) for each cluster. C) Graph showing the proportion of cells from each condition categorized by the 7 main labels 1) vCM (Cluster 0, 3, 5, 9 and 10), proliferative vCM (Cluster 2, 6 and 7), EPC(Cluster 1), EPC→DC (Cluster 4 and 11), aCM (Cluster 12) and non-differentiated cells (Cluster 13). D) A heat map of the top 15 differentially expressed genes between the 5 vCM clusters (Cluster 0, 3, 5, 9, and 10).

FIG. 15 (A-E). Pooled vCM by condition DEG and ORA. A) A heat map of cardiac genes of interest for all the vCM broken down by condition. B) Heat map of the top 15 differentially expressed genes between the vCM broken down by condition. Dot plots showing some of the ORA biological processes (BP) pathways that were significantly C) upregulated or D) downregulated in the vCM by conditions. E) Heat map of some of the differentially expressed genes contributing to enrichment in four GO BP pathways 1) Cardiac Ventricle Development, 2) Regulation of Canonical WNT Signaling Pathway, 3) Cardiac Conduction, and 4) Response to Calcium Ions.

FIG. 16. EHT Phenotypic maturation reveals enhanced tissue morphology and adult myofilament isoforms in the CM Tri-culture EHTs. A) Representative images of aActinin (green) and DAPI (blue) in EHT cryosections. B) Quantified sarcomere length and C) Cellular alignment in the EHTs as shown by the average coherency of phalloidin from EHT transverse cross-sections where 0 is randomly aligned and 1 is perfectly aligned. Quantitative rt-PCR of the D) MYH6/MYH7 ratio and E) MYH6, F) MYH7, G) GJA1, H) KCNJ2, I) ATP2A J) RYR2, and K) PLN normalized to the housekeeping gene EDF1 and the cardiac gene TNNT2. For (B) violin plot center dashed line represents the median and outer dashed lines represent the upper and lower quartiles of the distribution and CM+EPC(+SB) (n=162), CM+EPC→DC (n=174), CM Tri-culture (n=191), CM (n=145), CM (+SB) (n=125) sarcomeres were measured across three sections per EHT and 6 EHTs per condition across three independent experiments. For (C) the phalloidin coherency was determined for CM+EPC(+SB) (n=18), CM+EPC→DC (n=14), CM Tri-culture (n=15), CM (n=17), CM (+SB) (n=15) EHT cross-sections across 6 EHTs per conditions from three independent experiments. For (D-K) Each dot represents one technical replicate from n=3 EHTs per condition across three independent experiments.

FIG. 17 (A-E). Pooled proliferative vCM by condition DEG and ORA. A) A heat map of cardiac genes of interest for all the proliferative vCM broken down by condition. B) Heat map of the top 15 differentially expressed genes between the proliferative vCM broken down by condition. Dot plots showing some of the ORA biological processes (BP) pathways that were significantly C) upregulated or D) downregulated in the proliferative vCM by conditions. E) Heat map of some of the differentially expressed genes contributing to enrichment in four GO BP pathways 1) Cardiac Ventricle Development, 2) Regulation of Canonical WNT Signaling Pathway, 3) Cardiac Conduction, and 4) Response to Calcium Ions.

FIG. 18 (A-E). Epicardial engineered heart tissue proliferation and function. A) Experimental timeline for engineered heart tissue seeding where the red text represents experimental variables. B) Images of engineered heart tissues at day 29 or 30 in PDMS molds. C) Representative EHT images of WT1 (red), cTnT (green), and DAPI (blue). D) Representative images of cTnT (green), EdU (red), and DAPI (blue) where the white arrows point to proliferating CM. E) The quantified EHT CM proliferation via the percentage of cTnT+EdU+ cells. Scale 20 μm. In (E) the bar and error bars represent the mean±STDEV where each dot represents the average across three sections of one EHT and n=4 EHTs across two independent experiments for each condition was assessed. *p<0.05, **p<0.01, ***p<0.001, and ****p<0.0001.

FIG. 19. Epicardial cells enhance engineered heart tissue conduction while the CM triculture enhances electrochemical function and force. A) Representative EHT calcium transient (CaT) traces and quantified B) time to peak, C) maximum amplitude, and D) CaT upstroke velocity. Voltage optical mapping for the CM+EPC→DC (left), CM Tri-culture (middle) and CM (right) control showing E) representative ADP80 maps and F) quantified average APD80. G) Representative optical mapping maximum upstroke velocity maps and H) average maximum upstroke velocity. I) Optical mapping activation time maps showing signal propagation from where the point electrical stimulator was placed (end of the red arrow) to the other side of the EHT (point of the red arrow). The red arrow shows the distance and direction for which the conduction velocity was calculated across for these three EHTs. J) The quantified conduction velocity for all EHTs that showed an analyzable conduction pattern. Representative AP trace cropped to 1 second for the K) CM+EPC→DC and L) CM Tri-culture and M) CM conditions. N) The quantified twitch force for EHTs with a detectable force and those with no detectable force (N.D) as well as the O) twitch stress. For (BC) the data is represented as box and whisker plots with +marking the mean and n=12 EHTs per condition from three independent experiments. For (F), (H), and (J) each dot represents the average across all pixels for one EHT, the center line is the mean, and the error bars represent the STDEV. For (F) and (H) n=6 EHT across two independent experiments and for (J) n=6 EHTs for CM+EPC→DC and CM Tr-culture and n=4 for CM across two independent experiments. For (N—O) the bar graph represents the mean±SEM and CM+EPC(+SB) (n=10), CM+EPC→DC (n=10), CM Tri-culture (n=8), CM (n=12) and CM (+SB) (n=12) EHTs across three independent experiments were measured. *p<0.05, **p<0.01, *** p<0.001, and ****p<0.0001.

FIG. 20 (A-B). Engineered heart tissues and 2D co-culture fibroblasts content. A) Staining of cTnT (green-cardiomyocyte), TE7 (red-fibroblasts), and DAPI (blue-nucleus) in EHT sections from the CM+EPC(+SB) (top), CM+EPC→DC (middle) and CM Tri-culture (bottom) conditions. Scale 20 μm. B) Staining of cTnT (green-CM), TE7 (red-fibroblasts), and DAPI (blue-nucleus) in all four 2D co-culture conditions and a C) positive control where fully differentiated EPD-FBs were co-cultured with CM in 2D. Scale 50 μm.

FIG. 21 (A-C). M4 and F7-hiPSC Pluripotency and Karyotype. The full karyotype of the A) M4-hiPSC lines and the B) F7-hiPSC lines. C) Representative images of the M4-hiPSC line (left) and the F7-hiPSC line (right) stained for the pluripotency markers Oct4 (red) and SSEA4 (green) and a nuclear stain DAPI. Scale 50 μm.

FIG. 22, Generation of EHTs containing uniform mix of viable cardiomyocytes, fibroblasts, and macrophages. (A) Dimension of the negative EHT mold. (B) Cast PDMS EHT mold (Scale bar=1 cm). (C) The components of the cell-fibrin matrix cast into PDMS molds to generate macrophage-containing engineered heart tissues (Mac-EHTs). (D) Mac-EHTs maintained in a PDMS mold inside a tissue culture well (Scale bar=1 cm). (E) Representative images of cTnT (for cardiomyocyte, CM, red), TE7 (for fibroblast, FB, green) and DAPI (for cell nucleus, blue) stained EHT section (Scale bar=50 μm). (F) Representative images of CD68 (for macrophage, Mac, white), TE7 (for fibroblast, FB, green) and DAPI (for cell nucleus, blue) stained EHT section (Scale bar=50 μm).

### Tables

Table 1. Single-cell RNA sequencing vCM integration DEG and BP pathways analysis.
Table 2. Antibody dilution and catalog number.
Table 3. Single-cell RNA sequencing software details.
Table 4. q-RT-PCR Primers.

### DETAILED DESCRIPTION

Described herein are methods, a technology platform and protocols for the ex vivo generation of engineered heart tissues (EHTs) from human pluripotent stem cells (hPSCs) that advance the state of the art in modeling human myocardium. The platform encompasses directed differentiation protocols to derive essential cardiac lineages-including hPSC-derived cardiomyocytes (CMs), epicardial cells, and epicardial-derived fibroblasts- and integrates pathway modulation (TGFβ1 inhibition) to enhance ventricular specification, maturation, and proliferation of cardiomyocytes.

Assembly of these cell types in two- and three-dimensional fibrin gel formats yields tissues with improved calcium signaling kinetics, reduced pacing frequency, and increased axial contractile force compared to conventional hPSC CM monocultures.

Further, incorporation of hPSC-derived vascular progenitors can produce in situ microcapillary and mural cell network formation within the tissues, while addition and co-differentiation of uncommitted hPSC-myeloid progenitors can generate resident cardiac macrophages that confer immunocompetence and potential inflammatory responses to disease modeling cues in a tissue-specific manner. This integrated, developmentally informed EHT platform provides a physiologically relevant, immunocompetent model of human cardiac tissue with significant applications in disease modeling and drug discovery within the fields of cardiology and immunocardiology.

Also disclosed herein are methods of use and compositions for cell derivation; tissue and cell assembly strategies for EHTs, including all co-culture combination parameters, and applications of immunocompetent EHTs in modeling disease or injury insults characterized by inflammatory responses in the underlying pathology and tissue response, both natively and in the presence of putative therapeutic interventions.

hPSC-derived cardiomyocytes exhibit limited maturity in cellular function and physiology; the specific combination of co-culture with epicardial cells and epicardial-derived fibroblasts, all derived from hPSCs, significantly enhances maturation, ventricular specification, and functional performance in both two- and three-dimensional formats. The lack of native vascular and immune compartments in ex vivo cardiac models reduces tissue fidelity. Incorporation of hPSC-derived vascular and myeloid progenitors can address the above by providing perfusable microcapillary networks and generating resident cardiac macrophages, capable of recapitulating native inflammatory responses that drive fibrosis and pathology in the majority of cardiac diseases.

As used throughout this disclosure, the term “hiPSC” refers to human induced pluripotent stem cell-derived cardiomyocytes.

As used throughout the disclosure, the term “Cardiomyocytes” refers to the specialized, involuntary muscle cells of the heart's myocardium (wall).

As used throughout the disclosure, the term “3D Engineered Cardiac Tissue” refers to tissue and/or living structures generated through a range of fabrication methods, whether bioprinting, rotational jet spinning, manual seeding in casted molds, or other approaches. By the 3D fabrication of living biological tissues, 3D Tissue engineering models employ the use of 3D matrices to culture cells and create tissues that mimic morphology and function that occur naturally in vivo.

As used throughout the disclosure, the term “Fibroblast” means a type of cell that contributes to the formation of connective tissue.

As used throughout the disclosure, the term “maturation” refers to the final stages of differentiation and development where cells, tissues, or organs transform from a generalized, immature state into a more mature form. This process involves structural and biochemical changes that allow the tissue to attain its better functional capacity and structural integrity.

Current approaches to cardiomyocyte maturation include 1) mechanical stimulation, 2) electrical pacing devices to simulate environmental conditions of the mature heart, 3) media supplements to drive metabolic maturation, and/or 4) in vivo engraftment into animal hosts. These approaches increase complexity, cost, and compromise throughput and human physiological relevance. Strategies for vascularization or immune integration often rely on primary human cells such as umbilical vein endothelial cells or blood-derived macrophages that lack cardiac specificity. In contrast, the present disclosure includes a platform that provides a fully human, ex vivo, stem-cell-derived solution that simultaneously enhances cardiomyocyte maturation and cellular physiology while providing possible de novo vascular network formation and bona fide cardiac-specific inflammatory responses.

Accumulating evidence indicates that maturation limits cardiomyocyte (CM) proliferation and conversely, that proliferation occurs rarely in cardiomyocytes of the adult myocardium. As described herein, the methods show results that expand on that theory when co-culturing human induced pluripotent stem cell cardiomyocytes (hiPSC-CM) with epicardial cells and epicardial-derived cells in two-dimensional (2D) direct co-culture and 3D engineered heart tissues (EHT). In 2D direct co-cultures the percentage of proliferating CM increased at the same time as stark electrophysiologic improvements in calcium handling. Notably, no increase in single-cell CM force generation was observed, as determined using traction force microscopy. Single-cell transcriptomics revealed a significant shift in the bulk CM population in epicardial-CM co-cultures as characterized by more fetal-like myofilament isoforms but with enhanced pathways associated with electrochemical maturation and ventricular development. Co-culture of epicardial cells in the 3D-EHT model showed more limited proliferation, but a similar improvement in CM electrophysiologic function. These results indicate that certain aspects of CM maturation are enhanced, especially electrophysiologic properties, by epicardial cells while an embryonic phenotype is maintained (i.e., proliferation and myofilament isoforms). Consistent with developmental progression and the initiation of epicardial-to-mesenchymal transition (EMT), epicardial-derived fibroblasts were added to the EHTs containing epicardial cells. Interestingly, significant myofilament maturation and increased force generation by more than an order of magnitude is observed. Taken together, these results suggest that some aspects of CM maturation (i.e., electrochemical) occur during embryonic development when proliferation rates are relatively high and that sarcomere-associated mechanical maturation occurs at later developmental stages when proliferation has largely ceased.

Aa described herein it may be advantageous to first mimic embryonic development before the implementation of post-natal conditions. The role of epicardial and epicardial-derived cells on hiPSC-CM and their ability to promote hiPSC-CM maturation by mimicking embryonic heart development is discussed herein.

The epicardium is an epithelial layer that originates during fetal heart development in the proepicardial organ by E9.5 and migrates to cover the primary heart tube by E10.5-E12 in mice. Epicardial cells serve three major functions during embryonic development 1) secretion of key growth factors that drive CM proliferation and formation of the compact myocardium, 2) the main source of interstitial fibroblasts in the myocardium, the major extracellular matrix (ECM) secretors responsible for the cardiac skeleton and the mechanical integrity of this highly functional organ, and 3) the production of vascular smooth muscle cells that are responsible for driving coronary vascular formation. Epicardial cells during development undergo EMT to generate cardiac fibroblasts and vascular smooth muscle cells. The epicardium is so important for proper cardiac development, that murine models with deficiencies in the epicardium have thin ventricular walls, impaired coronary artery angiogenesis, and embryonic or immediate perinatal lethality. These findings indicate that the epicardium is essential for proper ventricular morphogenesis. Thus, this disclosure discusses the impact of epicardial and epicardial-derived cells on hiPSC-CM maturation by mimicking embryonic heart development.

In this disclosure fetal development was mimicked by creating co-cultures of CM with fetal-like hiPSC-derived epicardial cells to find that they simultaneously enhance hiPSC-CM electrochemical function, proliferation, and fetal sarcomeric expression profiles. When fibroblasts, cells essential for post-natal heart maturation, are then added to this multicellular experimental framework, proliferation is lost, electrochemical maturation is sustained, and sarcomere-mediated mechanical maturation is added. Thus, an intermediate condition on the road to CM maturation is defined wherein electrochemical maturation, but not mechanical maturation can occur with CM proliferation.

Co-cultures, tri-cultures and/or combinations of staged culturing of various cell types are used and described herein. For example, methods of producing engineered heart tissue (EHT) include providing a population of human pluripotent stem cell-derived cardiomyocytes (hiPSC-CMs) and contacting the hiPSC-CMs with epicardial cells (ECs) during a first culture period. The ratio of hiPCS-CMs to ECs may be approximately 1:1 or in some embodiments, hiPCS-CMs may outnumber the ECs in the first culture period or the ECs may outnumber the hiPCS-CMs in the first culture period. After the first culture period, the methods may further include introducing or generating within the culture one or more additional (non-) myocyte populations comprising one or more of epicardial-derived smooth muscle cells, endothelial cells, fibroblasts, or any combination thereof during a second culture period. The (non-) myocyte populations are provided in a temporally staged manner that mimics embryonic-to-fetal cardiac development, thereby promoting maturation, patterning, multicellular organization, or functional performance of the engineered cardiac tissue. The one or more of epicardial-derived smooth muscle cells, endothelial cells, fibroblasts, or any combination thereof may be provided to the co-culture of hiPSC-CM and EC in amounts that substantially equal, exceed and/or do not exceed the amount of hiPSC-CM and EC already in culture.

Use of the cardiomyocytes modeled according to one or more protocols described herein can be used for drug screening and disease modeling, wherein the disease comprises one or more of cardiomyopathy, cardiac channelopathies, cardiac metabolic disease, congenital heart disease, and/or neuromuscular cardiac disease, and/or can be used for screening candidate therapeutic agents affecting cardiac development or maturation, and/or studying lineage progression of epicardial cells and epicardial-derived stromal cells during cardiac development for example.

Also described herein are methods of using the EHT according to the protocols described here in to study human cardiac development. The study comprises evaluating multicellular interactions among cardiomyocytes, epicardial cells, fibroblasts, smooth muscle cells, and endothelial cells, further comprises evaluating lineage progression of epicardial cells into one or more epicardial-derived lineages. The EHT can be used in evaluating developmental stage-specific effects on cardiomyocyte proliferation, maturation, ventricular specification, conduction, calcium handling, or force generation. The EHT can further be used to model a disease associated with early cardiac development such as cardiomyopathy and other cardiac metabolic diseases. The EHT can be used as screening test agent for detecting the effect on cardiac development, lineage progression, multicellular signaling, calcium handling, conduction, force generation, fibrosis, vascular organization, or disease phenotype and/or for the evaluation as a test agent for prevention, reduction, or reversal of a phenotype associated with an early developmental cardiac disease.

In some embodiments, the disclosure provides an embryonic-to-fetal progression mimicry process in which hPSC-derived cardiomyocytes are first exposed to hPSC-derived epicardial cells during an early developmental stage, followed by staged introduction or in situ generation of additional support lineages including epicardial-derived smooth muscle cells, endothelial cells, and fibroblasts. In some embodiments, the staged progression is controlled by transient inhibition of TGFβ signaling to maintain an epicardial state, followed by a release phase that permits epithelial-to-mesenchymal transition and lineage progression. Such staged developmental sequencing may be implemented either by sequential addition of differentiated cell populations at prescribed time points or by directed differentiation of precursor populations already present in the culture.

In some embodiments, the engineered tissues are used to model human cardiac development, multicellular signaling, lineage progression, ventricular specification, conduction maturation, vascularization, stromal maturation, and diseases associated with early development, including cardiomyopathies, cardiac metabolic diseases, and other cardiac conditions and diseases.

As used herein, embryonic-to-fetal progression mimicry refers to temporal recapitulation in vitro of developmental cell-cell interactions in which epicardial signaling is presented before later stromal and vascular support lineages.

### EXPERIMENTAL

### Discussion

In one embodiment, murine hearts during E10, E12, and E17 were investigated and it was found that substantial amounts of CM maturation occur at this time. The effect of epicardial cells on ventricular CM in vitro was studied and it was found that they simultaneously spurred a more electrochemically functional but fetal-like phenotype characterized by increases in proliferation and expression of fetal myofilament isoforms. Single-cell sequencing revealed the upregulation of multiple signaling pathways, including WNT, that are implicated in CM proliferation and compact myocardium specification during development. The increased electromechanical function was validated in 3D-engineered heart tissues. To mimic the multicellularity present during development, fully differentiated epicardial-derived fibroblasts were added to the EHTs in a CM Tri-culture of EPCs and EPD-FB. These were maintained in TGF-b-inhibitors initially, followed by removal of the TGF-b inhibitor to allow for epicardial EMT and tissue compaction as in development. These tissues showed robust electrochemical maturation and force generation as well as increased adult myofilament isoforms. Taken together it was found that mimicking the multicellular progression of the fetal to late neonatal environment best supports hiPSC-CM functional maturation.

Epicardial cells are powerful mediators of hiPSC-CM electrochemical function. Enhanced calcium handling and AP parameters were seen in both 2D and 3D cocultures. Some of the potential mediators for this increase were determined to be ryanodine receptor 2 (RYR2), sarcoplasmic reticulum calcium ATPase 2 (ATP2A2 or SERCA2), and calcium voltage-gated channel auxiliary subunit b 2 (CACNB2 or CaVb2), as they were significantly upregulated in the EPC(+SB) co-culture. RYR2 is the dominant calcium channel that releases calcium from the sarcoplasmic reticulum. RYR2 has long been thought to be a mediator of CM maturation and in a recent mouse model, a RYR2-depleted murine model showed functional deficits, decreased presences of T-tubules, and decreased morphologic maturation (CM sarcomere length, CM area, and elongation). Therefore, the epicardial-induced CM RYR2 expression could point to the mechanism for morphologic maturation increases as well as more mature calcium handling. Conversely, ATP2A2 is responsible for clearing calcium from the cytosol and bringing it back into the sarcoplasmic reticulum. This could be related to the significant increase in CaT downstroke velocity seen in the EPC(+SB) co-cultures. CACNB2 encodes the predominant b-subunit for cardiac L-type calcium channel, CM specific KO of this gene leads to reduced L-type calcium current and vascular dysfunction leading to heart failure and embryonic lethality. L-type calcium currents are regulators of excitation-contraction coupling and this could serve as another mediator for enhanced conduction seen in the epicardial co-cultures. When RYR2 and ATP2A2 were looked at in the EHTs no changes were detected; this could be due to the loss of single-cell resolution in dense cultures and the challenge of finding a stable cardiac gene to normalize to in these highly heterogenous cultures. In the epicardial-derived co-culture calsequestrin 2 (CASQ2) was significantly upregulated. This is the most abundant calcium-binding protein of the sarcoplasmic reticulum. This could be responsible for increased calcium handling seen in these conditions as genetic over-expression of CASQ2 in hiPSC-CM has this effect.

Parallel findings of increased maturation and proliferation in this study are intriguing as CM proliferation and maturation are described as dichotomous. This may indeed be the case in vivo as murine data suggest CM proliferation does decrease as sarcomere length and cell area increase. It is important to note that the in vitro case is highly artificial in the way that it can exist without the epicardium since mouse models without the epicardium will not survive. It is also important to note that mouse embryonic CM maturation seen in this study occurs while CM proliferation is extremely high (~50%) meaning some amount of maturation does happen in vivo while CM are still in a highly proliferative state. The increases in proliferation we see in parallel with maturation could simply be a result of more accurately mimicking in vivo cardiac multicellularity. Additionally, the simultaneous increase in proliferation and calcium handling has been seen in previous in vitro hiPSC-CM and epicardial co-cultures. Taken together with the embryonic patch clamp data discussed above, there is evidence to suggest that proliferation and aspects of CM maturation, such as electrochemical function, do occur simultaneously in vitro. Unlike the 2D in vitro co-cultures, the 3D EHTs containing epicardial cells did not have enhanced proliferation after 29-30 days of co-culture. This was the first study that looked cocultures for this long and it is very likely that an increase in CM proliferation would be temporal and eventually subside as the 3D environments promotes enhanced CM maturation.

Without being bound by theory, the data in this disclosure suggests that mechanical maturation, through increased single-cell and tissue level force generation, as well as the robust expression of adult myofilament isoforms, likely occurs after CM-proliferation and some aspects of electrochemical maturation have occurred. This disclosure is the first to report that epicardial cells, before EMT, drive more fetal CM myofilament expression. Interestingly, epicardial-derived cells (in 2D) and epicardial-derived fibroblasts (in 3D) ameliorated this effect resulting in increased TNNI3 and MYL2 expression in 2D and an increase in the MYH7/MYH6 ratio in 3D. This finding fits with the progression seen in embryonic development where epicardial cells spur the expansion of CM which leads to the formation of the dense compact myocardium in fetal development and then myofilament isoform switching does not occur until postnatal maturation. The uncoupling of sarcomere length and myofilament isoform switching in the proliferation maturation dichotomy is a new finding and it is shown here that the longest sarcomere length in the CM+EPC(+SB) condition which was simultaneously proliferative and had the most fetal-like myofilament expression profile. The murine data supports this uncoupling as substantial increases in murine sarcomere length occur in vivo while the CM are still highly proliferative at a time point in embryonic development where fetal myofilament isoform expression is still dominant.

TFM data also suggests that myofilament isoform expression is not necessarily related to hiPSC-CM single-cell force generation. TFM reported no significant changes in single cell force generation even though fetal isoforms prevailed in the CM+EPC(+SB) co-culture and the CM+EPC→DC co-culture showed a distinct increase in adult myofilament genes TNNI3 and MYL2. Further, sarcomere length seemed to be a better predictor of increased force generation as the only conditions with a significantly different force were the CM+EPC(+SB) (~1.94 μm) and the CM(+SB) (~1.81 μm) conditions which had the highest and lowest sarcomere length respectively. The most mature electrochemical function was seen in the population determined to have a more fetal myofilament composition (CM+EPC(+SB)). Mimicking embryonic development can have a powerful impact on the maturation state of the CM, and that the resultant fetal phenotype is more electrochemically functional than its more “adult-like” control.

Epicardial cells have enormous effects on the phenotype of the hiPSC-CM and vice versa. In addition to the shift in calcium handling and myofilament genes discussed above, these results also pointed to multiple signaling cascades that were only upregulated in the epicardial co-cultures. Among these, the WNT signaling cascade is of interest because of the role it has been seen to play in spurring CM proliferation in vivo and in vitro. WNT signaling was increased in both the vCM and the EPCs from the co-culture conditions but not in the mono-culture conditions for either cell type. Additionally, CM proliferation seen in vivo is also coupled with the appearance of the compact myocardium, a portion of the myocardium lacking in epicardial KO mice models. Interestingly, small molecule WNT activation has also been used to induce expression of the compact myocardium markers HEY2 and FZD2 in hiPSC-CM. These markers, along with increased CM proliferation, were both significantly increased here in the CM co-cultured with epicardial cells. This indicates WNT as a potential mechanism for epicardial-induced proliferation and compact myocardium specification. In development, the growth factor IGF2 has also been seen to mediate CM proliferation/ventricular compaction. Though IGF2 was not a DEG at the transcript level in the single cell sequencing, multiple insulin-like growth factor binding proteins were enriched in the CM+EPC(+SB) conditions EPCs leading to pathway enrichment of insulin-like growth factor receptor signaling. Other studies have used protein level assays to detect the presence of IGF2 in EPC and CM co-cultures and have determined that with the blocking of IGF2 receptors epicardial cells no longer increase CM proliferation. This mechanism could possibly converge on WNT signaling as well since IGF2 has been seen to activate WNT5A expression and the WNT signaling pathway in cancer cells. WNT5A is a DEG in that was upregulated in the vCM of our CM+EPC(+SB) co-cultures. This indicates that WNT5A could be explored as the potential mediator of IGF2 and WNT-induced CM proliferation perhaps as a more refined and less variable approach to promote CM proliferation, compact myocardium specification, and potentially even electrochemical functionality, than the use of EPC co-culture.

Taken together, this study is the first to describe the robust electrochemical maturation conferred by pure populations of epicardial cells. It also holds significant implications for the field of cardiac tissue engineering as it identifies the embryonic phase of CM maturation as a unique and perhaps critical component of hiPSC-CM maturation. As developmental biology was mimicked to determine the pathways involved in CM-specification to yield robust hiPSC-CM differentiation protocols the temporal modulation of CM embryonic maturation cues followed by post-natal cues could be envisioned for the realization of a fully function adult phenotype in hiPSC-CM.

### Materials and Methods

### Human Induced Pluripotent Stem Cell Lines

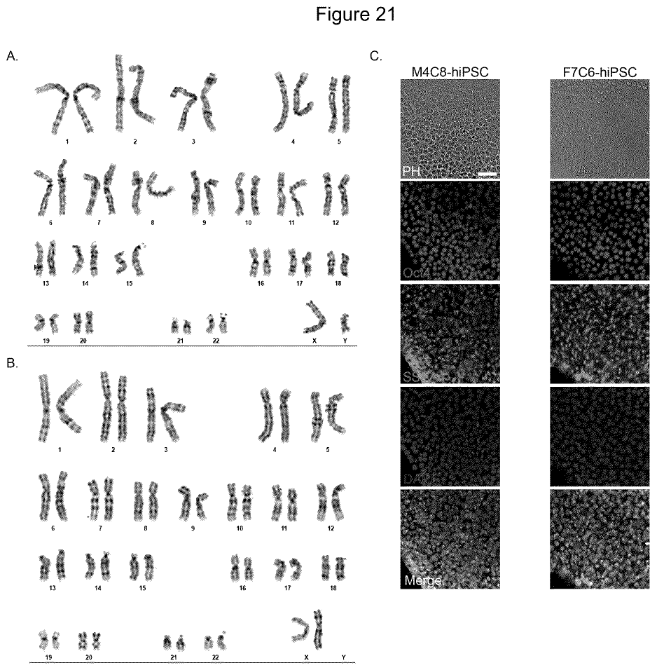

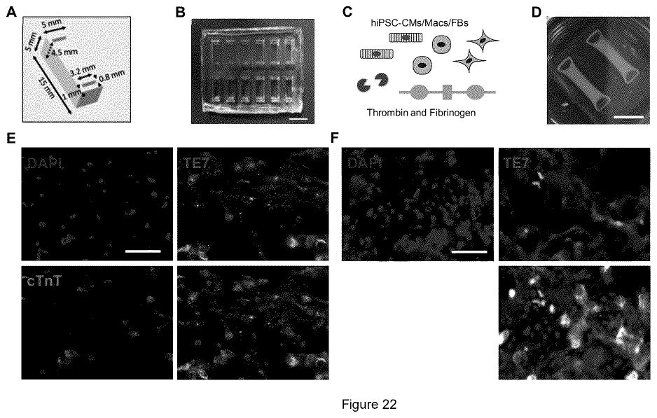

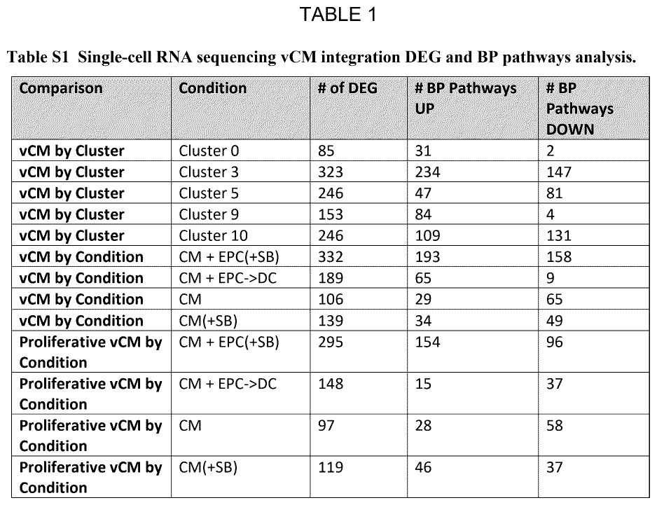

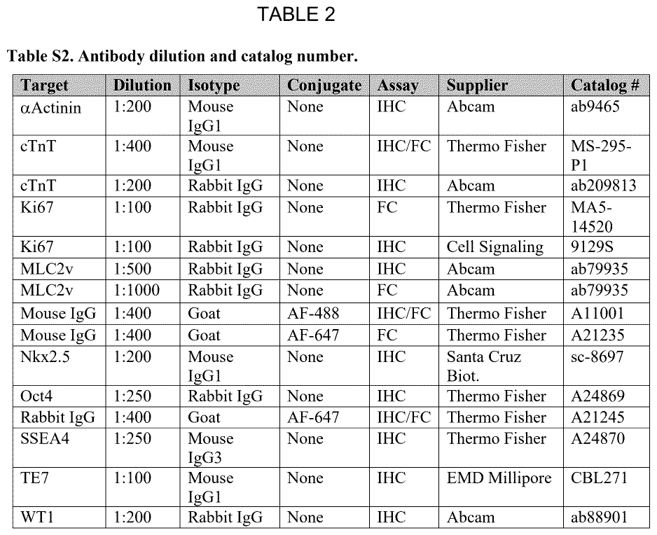

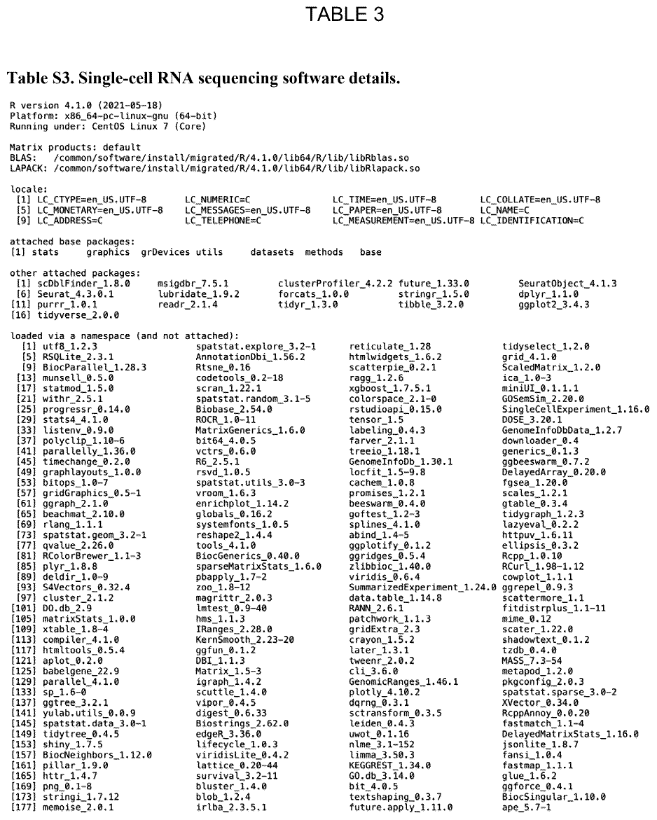

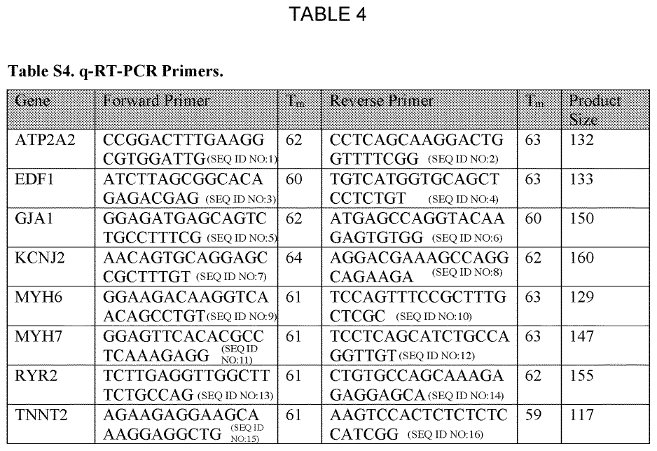

Three hiPSC-lines were used for these studies. All of the lines were derived from left ventricular cardiac fibroblasts—two lines from female donors, CCND2-hiPSC and F7C6-hiPSC, and one line from a male donor M4C8-hiPSC. Sendai virus was confirmed to be depleted via qPCR for two passages. A full karyotype was performed on the lines after Sendai virus depletion was confirmed and pluripotency was seen to be high and the karyotype showed no abnormalities (FIG. 21).

### Human Induced Pluripotent Stem Cell Maintenance

The hiPSCs were maintained in a 6 well plate coated with 8.68 μg/cm², or 0.5 mg per well, Cultrex-reduced growth factor (RGF) (R&D Systems 3433-005-01) in 2 mL of mTesRTM1 (STEMCELL 85850) and passaged using ReLesSRTM (STEMCELL 05872) upon reaching 70-80% confluency as follows. Exhausted media was aspirated, and cells were washed with 1 mL of DPBS without calcium or magnesium. Then, 1 mL of room temperature ReLesSRTM was added to each well and incubated for 45 seconds. The ReLesSRTM was aspirated, and the plate was put into the incubator at 37 °C for 7 minutes. Warm mTesRTM1 was added to the well and the edges of the plate were gently tapped to release and break the hiPSC colonies from the bottom of the plates. The cell suspension was diluted and transferred into a new 6 well-plate.

### CM Differentiation and Purification

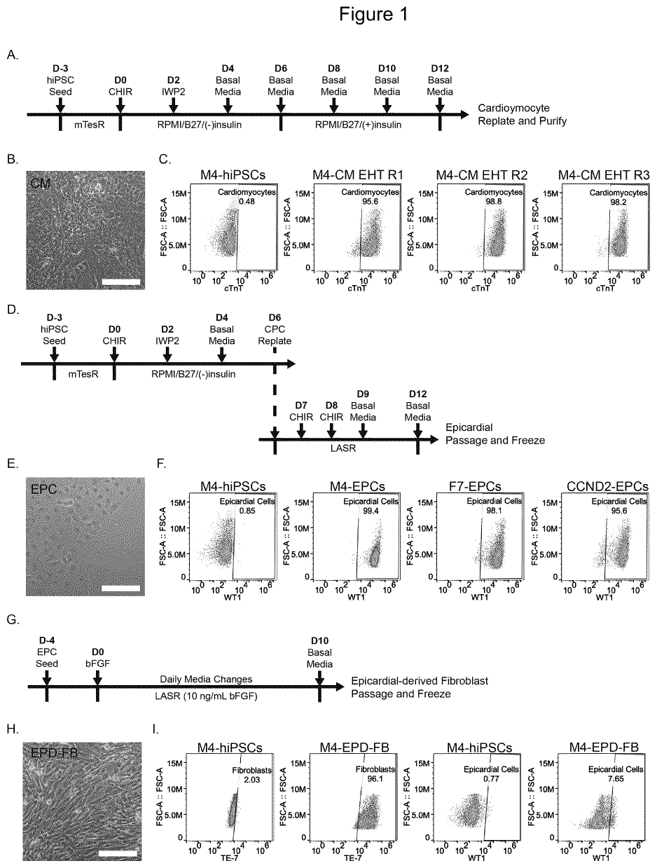

The hiPSCs were maintained until reaching 70-80% confluency and singularized using AccutaseÒ solution (Millipore Sigma A6964) as follows. Exhausted media was aspirated, and the cells were washed with 1 mL of DPBS without calcium or magnesium, and 1 mL of room temperature AccutaseÒ solution was added to each well and incubated at 37 °C for 8 minutes. The hiPSCs were singularized by pipetting them up and down with a P1000 micropipette and quench using mTesRTM1. The cell suspension was transferred to a conical tube, mixed, and counted on a hemocytometer using a 1:1 dilution of 0.4% trypan blue. The hiPSCs were seeded onto 8.68 μg/cm2 Cultrex-RGF at a density of ~132,000 cells/cm², or 0.5×106 cells per 12 well, into 1 mL of mTesRTM1 with 5 μM Rock Inhibitor (VWR 103538-728). The exhausted media was replaced with 2 mL of fresh mTesRTM1 until the cells reached 97-100% confluency (2-4 days). All media changes unless otherwise specified were a volume of 2 mL. Upon reaching confluency, defined as day 0 of the differentiation, the media was replaced with RPMI/B27 (-ins) (Fisher A1895601) containing 6-8 μM CHIR99021 (Sigma SML1046). The exact CHIR99021 concentration for mesoderm specification depends on the hiPSC-line. After 48 hours, on day 2 of differentiation, the media was replaced with RPMI/B27 (-ins) containing 7.5 μM of IWP2 (Tocris 686770-61-6). After another 48 hours, on day 4 of differentiation, the media was replaced with fresh RPMI/B27 (-ins). On day 6, and every two days until day 13 of differentiation, the media was replaced with fresh RPMI/B27 (+ins) (Thermo 17504001). The timeline is depicted visually in the supplemental materials (FIG. 1). To aid in lactate purification, the CM were replated at a 1:2 ratio onto 8.68 μg/cm2 Cultrex-RGF coated 12 well-plates. Briefly, each well was washed with 1 mL of DPBS without calcium or magnesium then 1 mL 37 °C. 0.25% trypsinEDTA solution was added to each well and incubated at 37 °C for 20 minutes. The CM were singularized by pipetting with a P1000 micro pipettor and then quenched with 2 mL of RPMI/B27 (+ins) with 20% FBS. The cell suspension was transferred to a conical tube and centrifuged at 200 g for 5 minutes. The supernatant was aspirated from the cell pellet and the cells were resuspended in RPMI/B27 (+ins) containing 10 μM Rock Inhibitor. On day 14, the replated cells are given 1 mL of RPMI/B27 (+ins). The CM were purified using lactate purification media (DMEM no glucose with 4 mM lactate) (Thermo 11966025, Sigma L7022). On day 15 the media was replaced with 1 mL lactate purification medium. On day 17 the cells were washed with 1 mL of DPBS without calcium or magnesium, and the media was replaced with 1 mL lactate medium. On day 19 the cells were washed with DPBS without calcium or magnesium again and given fresh RPMI/B27 (+ins) every three days until replated for 2D coculture or engineered heart tissue seeding. Differentiation quality controls show cTnT>95% (FIG. 1B-C).

### Epicardial Differentiation

The epicardial differentiation protocol was adapted from a previous publication and visually depicted in the supplement (FIG. 1D). The CM differentiation as detailed above is followed until day 6. On day 6, the cardiac progenitor population is replated onto a gelatin coated 6-well plate at a density of 30,000 cells/cm². Briefly, each well was washed with 0.5 mL of DPBS without calcium or magnesium then incubated in 0.5 mL of Accutase® for 8 minutes at 37 °C. The cardiac progenitors were singularized using a P1000 micro pipettor and then quenched with 0.5 mL LASR media (DMEM/F12 Advanced, 1.25% GlutaMAX, 60 g/mL Ascorbic Acid). The cell suspension was transferred into a conical tube and counted using a hemocytometer with a 1:1 dilution of 0.4% trypan blue. The cells were plated onto a gelatin coated 6-well plate in LASR media with a 5 μM Rock Inhibitor. All media changes were a volume of 2 mL for this differentiation process. On days 7 and 8, the medium is replaced with LASR containing 2-3 μM CHIR99021. The exact CHIR99021 concentration for epicardial specification depends on the hiPSC-line. Starting on day 9 the cells are given fresh LASR media every 2-days until day 12. On day 12 the epicardial cells (EPCs) are passaged or cryopreserved at 1 million cells per vial in epicardial freezing medium (LASR media with 30% FBS, 10% DMSO, 5 μM Rock Inhibitor, 2 μM SB431542). Epicardial cells were used between passages 2 and 5 in these experiments and were all cryopreserved before use. Briefly, the epicardial cells can be thawed onto a gelatin-coated 6-well plate into LASR media with 1% FBS, 5 μM Rock Inhibitor, and 2 μM SB431542. The media was changed with LASR media containing 2 μM SB431542 daily until the cells reached 80-90% confluency. At this point, the cells were passaged or used for a co-culture experiment. The epicardial cells were passaged at a 1:3-1:6 ratio onto a gelatin-coated plate using Versene (Thermo 15040066). Each well was washed with 1 mL of DPBS without calcium or magnesium and then incubated in 1 mL of Versene for 8 minutes at 37 °C. The Versene was aspirated and 1 mL of fresh LASR media with 1% FBS and 2 μM SB431542 was added to the cell layer. The cells are broken into clumps using a P1000 micro pipettor, transferred into a conical tube, diluted, and replated into a new gelatin-coated 6-well plate. The differentiated epicardial cells had a distinct epithelial cobblestone morphology (FIG. 1E) and were determined to be 3 95% pure via flow cytometry for WT1 (Wilms tumor 1) (FIG. 1F).

### Epicardial-Derived Fibroblast Differentiation

The epicardial-derived fibroblast differentiation is shown in FIG. 1G. At day 12 of epicardial differentiation the epicardial cells were passaged twice, as detailed above without cryopreservation, and then grown until they reached 100% confluency in LASR media with 2 μM SB431542. At this point, the medium was replaced with LASR containing 10 ng/ml bFGF (R&D Systems 233-FB-010) daily for 10 days. At day 10, the cells were replated at a 1:6 ratio onto tissue culture plastic into fully supplemented FibroGRO (Millipore Sigma SCMF002). To passage, the epicardial-derived fibroblasts (EPD-FBs) were washed, with 1 mL of DPBS without calcium or magnesium then incubated in 1 mL of Accutase® for 20 minutes at 37 °C. The cells were broken up using a P1000 micro pipettor and plated into FibroGRO on tissue culture plastic at a 1:6-1:18 ratio. The FibroGRO medium was replaced every 2 days with fresh media until the cells reached 70-90% confluency. The EPD-FBs were used in engineered heart tissue experiments between passage 3 and passage 7. The fibroblasts had an elongated stomal cell morphology, were 3 95% pure via flow cytometry for TE7, and had less than 10% remaining WT1-positive cells (FIG. 1H-I).

Direct Co-Culture of CM with Epicardial Cells

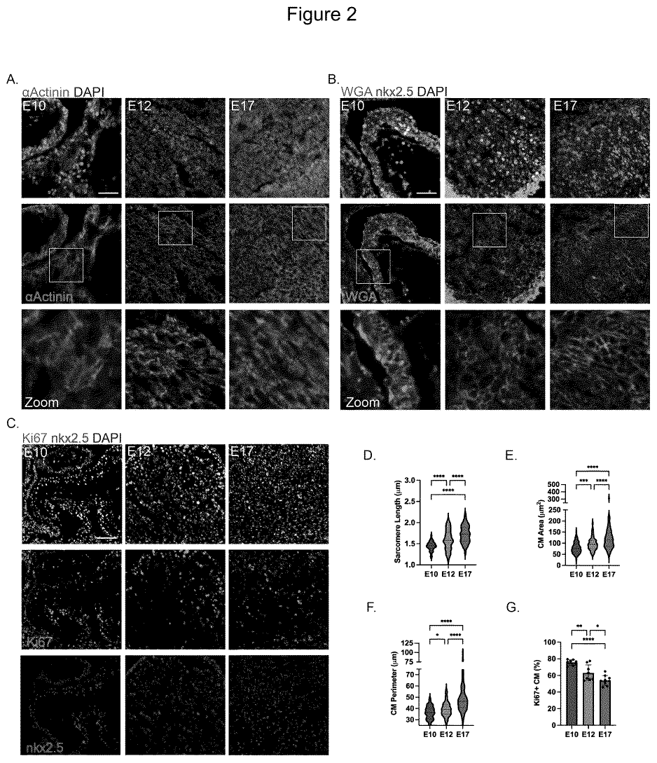

CM and EPC co-cultures were seeded in a layered fashion to allow for maximum and consistent CM attachment. First, the plates (12 and 48 well-plates) were coated overnight at 37 °C with 17.36 μg/cm² of Cultrex-RGF. On day 26 of CM differentiation the CM were washed with 0.5 mL of DPBS without calcium or magnesium and 0.5 mL 37 °C. 0.25% trypsin-EDTA solution was added to each well and incubated at 37 °C for 20 minutes. The CM was singularized by pipetting with a P1000 micro pipettor and then quenched with 1 mL of RPMI/B27 (+ins) with 20% FBS and 5 μM Rock Inhibitor. The cell suspension was transferred to a conical tube and counted with a hemocytometer with a 1:1 dilution of 0.4% trypan blue. The cells were centrifuged at 200 g for 5 minutes and plated at a density of 75,000 cells/cm² in LASR media containing 10 μM Rock Inhibitor. On day 27, after the CM had fully attached, the epicardial cells were seeded on top of the CM at a density of 25,000 cells/cm² in LASR containing 5 μM Rock Inhibitor (CM+EPC→DC) and 2 μM SB431542 (For the CM+EPC(+SB) condition only). On day 27 the CM-only conditions were given LASR containing 5 μM Rock Inhibitor (CM) and 2 μM SB431542 (For the CM (+SB) condition only). The media was changed to fresh LASR with or without 2 μM SB431542 on day 28 and every 2 days until day 35 of CM differentiation (8 days of co-culture total). To label newly synthesized DNA co-cultures controls that were fated for flow cytometry proliferation assessment were given LASR containing 10 μM 5-ethynyl 2′-deoxyuridine (EdU) for 24 hours before singularization and fixation. On day 35, the CM-only controls and co-cultures were assessed and processed in various ways as detailed below. The 2D co-culture timeline is depicted in FIG. 2A.

### Engineered Heart Tissue Seeding

The EHT negative mold was printed on a Stratasys J750 PolyJet 3D Printer using digital acrylonitrile butadiene styrene (ABS). The ABS negative mold was thoroughly cleaned, oxygen plasma treated for 10 minutes (PDC-32G, Harrick Plasma), and treated with silane vapors for 24-hours in a desiccator vacuum. Sylgard-184 polydimethylsiloxane (PDMS) precursor was mixed at a 1:10 mass ratio with a PDMS curing agent and cast onto the ABS negative mold. The curing PDMS was degassed for 1 hour at room temperature and cured overnight at 500 C. The resultant PDMS mold contains 12 rectangular EHT culture wells and each EHT well contains two posts. The PDMS mold was further cured at 50 °C for 1 week. The mold was then cut into six 2-mold squares that were sonicated in 70% ethanol for 30 minutes and autoclaved at 121 °C for 25 minutes. The sterile two EHT well PDMS squares were transferred into a sterile petri dish and oxygen plasma was treated for 2 minutes. The PDMS EHT molds were then transferred to a 12-well plate and treated with 0.5 w/v % Pluronic F-127 (Sigma P2443) for 30 minutes at room temperature. At day 24 of CM differentiation the purified hiPSC-CM were dissociated in 0.25% Trypsin-EDTA and combined with EPCs or EPCs and EPD-FBs (CM Tri-culture) that were singularized in Accutase®. The purity of the CM used in the EHTs were assessed to be ≥95% pure via flow cytometry for cardiac troponin T (cTnT) (FIG. 1). The hiPSC-CM and EPCs or EPCs and EPD-FBs were combined at a 3:1 ratio at a density of 10×106 cells/mL in a fibrin gel mixture containing 20 mg/mL fibrinogen from human plasma (Sigma F3879), LASR with 25 μM Rock Inhibitor and 100 U/mL thrombin from human plasma (Sigma 605190) at a 6:3:1 ratio. Each PDMS EHT culture well was made with 100 μL of fibrin gel cell suspension which instantly formed a gel at room temperature upon the addition of thrombin. For the CM-only control conditions, 7.5×106 CM/mL of fibrin gel mixture was used so that all conditions contained the same number of CM. Two EHTs of the same condition were cultured per well of a 12-well plate. The EHTs were cultured in LASR with 10 μM Rock Inhibitor, 20 μg/mL aprotinin (Sigma 10981532001), and (+/−) 2 μM SB431542 for 24 hours after seeding. The EHTs were then given fresh LASR media with 20 μg/mL aprotinin (+/−) 2 μM SB431542 every two days until assessment at 29-31 days after EHT seeding. The EHTs fated for immunohistochemical (IHC) analysis were treated with 10 μM EdU for 24 hours before fixation and processing as detailed below.

### Calcium Transients of Direct Co-Cultures, Transwell Co-Cultures and EHTS

On day 35, the 2D co-cultures and controls that were plated into 12 well-plates were incubated with 5 μM Rhod-2AM calcium-sensitive dye in LASR medium for 30 minutes at 37 °C. For the EHTs calcium was assessed 29-31 days after EHT seeding. The EHTs were kept in the PDMS mold and incubated with 5 μM Rhod-2AM calcium-sensitive dye in LASR medium for 30 minutes at 37 °C. The dye was aspirated and replaced with Tyrode's Salt Solutions with 1 g/L sodium bicarbonate (TSS) (Sigma Aldrich T2145). The 2D cells or EHTs were equilibrated in TSS for 30 minutes at 37 °C and then imaged on the TxRed channel with a 30-millisecond exposure and gain of 1.0 at 10× magnification on a fluorescent microscope (Leica Microsystems). Three 20-second videos per well or EHT were taken, with n=3 wells per condition or n=3-4 EHTs per condition for each replicate. Videos were taken at a frame rate of 7 frames/s. The co-cultures and EHTs were paced using a platinum electrode at 1 Hz with a voltage of 7 V and pulse duration of 0.02-milliseconds. The calcium transient data acquired was processed in ImageJ by importing the images and converting them into image stacks. Three regions of interest within each video were selected and the Z-axis profile of fluorescent intensity versus time was exported as a text file. The text files were analyzed using an in-house MATLAB code to determine the inter-spike-interval(s) of the calcium peaks, the time to peak(s), the maximum amplitude (F/F0) of the calcium peak as well as the average upstroke and downstroke velocities (F/F0/s). Any videos acquired in which the inter-spike-interval was less than 0.95 or greater than 1.05 were excluded due to insufficient pacing.

### Patch Clamp of CM

Patch clamp was done for the CM+EPC(+SB) and the CM (+SB) control only. The direct 2D co-cultures were replated at a density of ~10,000 cells/cm² onto 35 mm TC-treated petri dishes coated with 17.36 μg/cm² Cultrex-RGF. After 24 hours patch clamp measurements of the replated CM were taken. The patch clamp and AP recording method is described in a previous publication66. In brief, an Axopatch-200B amplifier (Molecular Devices, Foster City, USA) was used to record APs by a ruptured whole-cell current-clamp technique. For AP recordings, pipettes were filled with (in mmol/L) 120 potassium gluconate, 20 KCl, 5 NaCl, 5 HEPES, 0.02 EGTA, 0.05 CaCl2, and 5 MgATP (pH 7.2, [Ca22+]i≈100 nmol/L). The extracellular bathing solution contained (in mmol/L) 140 NaCl, 5.4 KCl, 1 MgCl2, 10 HEPES, 1.8 CaCl2, and 5.5 glucose (pH 7.4). Pipette resistances were ~3 MΩ. The recordings were low pass filtered at 10 kHz and digitized at 20 kHz.

### Traction Force Microscopy of 2D Co-Culture CM

Traction force microscopy (TFM) was executed by the construction and micropatterning of polyacrylamide gel constructs as previously described13,89-91. Briefly, stamps were made from PDMS from a master silicon wafer that was fabricated using photolithography. The stamps are rectangular arrays containing ~20 μm×140 μm features used to micropattern islands that can be coated with protein for cell adhesion. For sterilization, the stamps were sonicated with 70% ethanol for 30 minutes then coated with ~123 μg of Cultrex-RGF per substrate diluted in DMEM/F12 with HEPES and left at 37 °C overnight. Next, 15 mm glass coverslips were rinsed with acetone and ethanol and then plasma treated for proper cleaning. The Cultrex-RGF stamps were then placed feature side down on the plasma-treated coverslips. The polyacrylamide gel was created and bound to a 25 mm glass coverslip as follows. The 25 mm glass coverslip was cleaned and treated with UV for 8 minutes. The glass was functionalized with 3% silane solution for 5 minutes. The prepolymer polyacrylamide gel solution was composed of 10/0.13% w/v acrylamide/bisacrylamide that was doped with 2% v/v 0.2 μm diameter red fluorescent microspheres. After the solution was degassed, 0.002/0.05% w/v initiators tetramethylethylenediamine/ammonium persulfate, 0.005% w/v N-hydroxysuccinimide ester, and 0.014% 1 M HCl were added to the gel. Then 10 μL of gel solution was pipetted onto the center of the silane-treated glass coverslip. The Cultrex-RGF coated coverslips were placed pattern side down onto the polyacrylamide gel solution and polymerized at room temperature for 1 hour. The polyacrylamide gel was rehydrated and the Cultrex-RGF coverslip was removed. The Young's modulus of the polymerized polyacrylamide gel was previously determined to be 13.5 kPa90. At day 35 of the 2D direct co-culture experiments the co-cultures were singularized using 0.25% trypsin-EDTA as previously described and plated at a density of 35,000 cells per micropatterned polyacrylamide gel substrate into LASR medium containing 10 μM Rock Inhibitor. The following day the cells were given fresh LASR media. After 48 hours of recovery on the substrates, TFM measurements were acquired. Videos were taken on an inverted microscope in a CO₂ controlled chamber at 37 °C CM beat rate was controlled using 1 Hz electrical stimulation with a 40 ms bipolar pulse with a MyoPacer external field stimulator (IonOptix). Only micropatterned hiPSC-CM that were paced were imaged for these studies. Both brightfield and fluorescent images were acquired of the micropatterned hiPSC-CM at a frame rate of 30-100 frames per second. The images were taken for 3 seconds so that three consecutive contractions could be imaged in each video. Following image acquisition, the cells were lysed using sodium dodecyl sulfate, and cell free images of the fluorescent bead layer were acquired. The TFM measurements were analyzed in ImageJ as previously described13,89-91. The cell induced bead displacement between the cell-attached and cell-free images at each time point was calculated using a particle image velocimetry algorithm in ImageJ. To generate a field of substrate traction stress vectors, an unconstrained Fourier-transform traction cytometry algorithm⁹² was applied to the field of cell-induced bead displacements with a regularization factor of 1×10-9 and a Poisson's ratio of 0.5. Substrate traction stress vectors acting on point n are defined by

$${T}^{n} = {T}_{x}^{n} {e}_{x} + {T}_{y}^{n} {e}_{y} , where {T}_{i}^{n}$$

is the traction stress acting in the ith direction (i=x, y), and eᵢ is the unit vector in the ith direction. Substrate traction forces acting at point n were taken as the substrate traction stress vector Tn multiplied by the area α″ over which Tn acts. At the cell-gel interface, substrate traction forces are balanced by internal cell forces fⁿ such that fⁿ+Tⁿαⁿ=0. Substrate traction forces in turn are defined as is

$${f}^{n} = {f}_{x}^{n} {e}_{x} + {f}_{y}^{n} {e}_{y} , where {f}_{i}^{n}$$

is the internal cell force acting on point n in the ith direction. The total internal cell force fi acting in direction i was summed around the midline of the cell, such

$$2 {f}_{i} = {\sum }_{n} {f}_{i}^{n} {r}_{i}^{n} / \vert {r}_{i}^{n} \vert , where {r}_{i}^{n}$$

is the distance in the ith direction between the cell midline and the location n of the traction force. The force of contraction was defined as the difference in internal cell force between the cell at its maximum contraction and the uncontracted state. The contraction force was calculated for three consecutive contractions of the cell and then averaged.

### Optical Mapping of EHTS

The EHT device underwent a 20-minute immersion in a 10 μM voltage-sensitive dye di-4-ANEPPS solution prepared in TSS. Subsequently, the dye mixture was replaced with fresh TSS. After a stabilization period of 2-5 minutes, EHT were excited using two continuous-excitation green lasers (532 nm, 1 W; Shanghai Dream Lasers Technology, Shanghai, China). Fluorescence intensity was captured for 10 seconds using high-resolution cameras (14-bit, 80×80-pixel, Little Joe, RedShirt Imaging, SciMeasure, Decatur, GA) at a rate of 500 frames per second13,89-91. Pacing at 1 Hz was conducted through a bipolar electrode, and optical mapping recordings were taken during this pacing and filtering using a bandpass filter 3-30 Hz. The duration of the optical APs (APD) was assessed at 80% repolarization. From this 2D APD maps were generated to illustrate the spatial dispersion in APD. We also quantified activation times (AT) at the peak of the derivative of voltage over time (dV/dt max), and calculated local conduction velocity (CV) as described previously.

### Twitch Force and Stress Measurements of EHTS

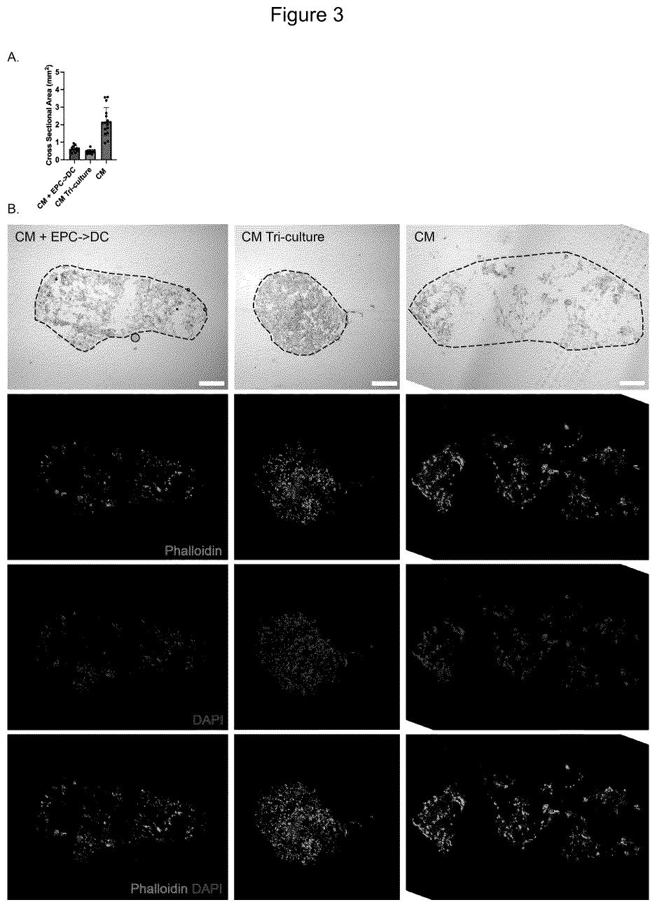

After 29-31 days in culture the EHTs were taken off the PDMS posts and mounted at their initial length on needles on the Mach-1 Micromechanical Tissue Tester (Biomomentum) using a 10 g force transducer. The EHTs were paced using a platinum electrode at 1 Hz with a voltage of 7-9 V and pulse duration of 0.02s. The force measurements were acquired for 20s at strains of 0, 5, 10, 15, and 20%. Using MATLAB a low pass frequency filter was used to remove noise from force data and the average twitch force was calculated. The maximum force for each EHT at any given strain was reported as the maximum twitch force. Cryosections of the EHTs were used to determine the average cross-sectional area and the twitch stress was calculated by dividing the average twitch force by the average cross-sectional area of the cryosections (FIG. 3).

### Murine E10, E12, and E17 Heart Harvesting, Sectioning, and Staining

All mouse experiments were performed using the outbred CD-1 mouse line. Upon mating, the detection of a mucus plug was considered day 1 of embryonic development. At the indicated time points, pregnant dams were euthanized, and embryos were extracted from the uterus. For the embryonic day (E) 10 and E12 whole embryos were used, while for E17 samples the heart was first excised from the embryo. For E17, hearts were immediately embedded in Tissue-Tek O.C.T. For E10 and E12 samples, embryos were fixed in fresh 4% paraformaldehyde (PFA) in PBS for 1-1.5 hours in a 12-well plate at room temperature, followed by a 3×15 min wash in PBS. Afterward, embryos were placed in a 5% sucrose in PBS solution at 4 °C until the tissue sunk to the bottom of the plate, followed by 10%, 15%, and finally 20% sucrose solution. E10 and E12 embryos were embedded in a 7.5% gelatin in a sucrose solution. Tissue blocks were sectioned into 4 μm slices. E17 samples were fixed in 4% PFA solution for 10 minutes at room temperature, followed by two washes for 5 minutes in PBS and then directed to permeabilization. For E10 and E12 samples, prewarmed PBS was used to wash off gelatin. Slides were permeabilized in 2% BSA, 0.5% Tween-20 in PBS for 10-15 minutes and blocked in 2% BSA, 0.1% Tween-20 for 1 hour both at room temperature. Slides were incubated overnight at 4 °C in a blocking solution with primary antibodies of nkx2.5, alpha-actinin, Ki67, and Wheat Germ Agglutinin (W11261, Invitrogen, 1:200 dilution). Antibody catalog number and dilution in Table 2. After four times washing for 2 minutes in PBS, slides were incubated in secondary antibodies at room temperature at 1:400 dilution and DAPI for 1 hour. The images were taken with a Zeiss upright microscope equipped with an AxioCam MRm camera, using the 40× and 20× objectives, and processed using Fiji software. CM size was quantified using Fiji by tracing the area of individual CM. Around 120 CM sizes were measured for each time point. The percentage of proliferative ventricular CM was quantified using Cellprofiler. Sarcomere lengths were measured using the Fiji plugin SarcOptiM. At least 100 CM sarcomeres were measured for E12 and E17 time points, and 90 were measured for E10 samples.

### Flow Cytometry for CM Proliferation, Phenotype, and Differentiation Quality Control

For proliferation assessment, the co-cultures were treated with EdU for 24 hours before harvesting on day 35. For co-culture proliferation and ventricular phenotype assessment, the cocultures were singularized using 0.25% Trypsin-EDTA for 20-minutes at 37 °C. Quality control samples of day 26 CM, EPD-FBs, and aLVCF were all singularized using the methods describes above. Once singularized, the cells were spun down at 200 g for 5 minutes, the supernatant was poured off and the cells were resuspended in 1% PFA in PBS for 20 minutes. After 20 minutes the cells were spun down at 200 g for 5 minutes, the fixative was removed, and the cells were resuspended in −20 °C 90% v/v Methanol. The samples were stored in the freezer at −20 °C. until staining for flow cytometry. For staining, the cells were strained with a 70 μm cell strainer (Corning 352350) to remove any large clumps. ~300,000-1,000,000 cells per sample were separated into individual conical tubes and the methanol was washed off the cells by adding 2 mL of flow buffer (PBS with 5% BSA and 0.2% Triton-X-100) spinning down the cells at 200 g for 5 minutes, pouring off the supernatant, then repeating this process once more. After the second wash, a P200 micropipette was used to remove as much residual flow buffer from the pellet as possible, and then the cells were either resuspended in 100 μL of Click-It EdU reaction mixture with Cy5-Azide for 30 minutes at room temperature (for proliferation assay only) or 100 μL of primary antibody dilution for 1 hr at room temperature. For the proliferation assay, after the Click-It reaction, the cells were washed twice with 2 mL of flow buffer as described above and then resuspended in 100 μL of primary antibody dilution for 1 hr at room temperature. Following the primary antibody incubation, the samples were washed twice with flow buffer as described above and then resuspended in 100 μL of secondary antibody solution. The cells were incubated at room temperature for 30 minutes and then washed twice with flow buffer. Following staining the cells were resuspended in ~300 μL of flow buffer and data was acquired for 10,000 events within the defined cell population using a BD Accuri C6 flow cytometer. The antibody dilutions and catalog number used for flow cytometry can be found in Table 2.

### CM Morphology Assessment

At the endpoint of co-cultures, day 35, the 2D co-cultured were using 0.25% Trypsin-EDTA for 20 minutes at 37 °C and replated onto 17.36 μg/mL Cultrex-RGF coated glass chamber slides at a density of ~10,000 cells/cm². After 24 hours the cells were fixed in diastole using 4% paraformaldehyde in PBS with 90 mM KCl, 4 mM EDTA, and 4 mM MgCl2 for diastolic arrest of the CM. The chamber slides were stored in PBS with 0.1% sodium azide at 4 °C until staining. To stain, samples were permeabilized using 0.2% Triton-X-100 for 1 hour, blocked in BGST (50 g/L BSA, 10 g/L glycine, 2% Goat Serum, 0.1% Triton-X-100) for 2 hours and then incubated with anti-aActinin antibody for 3 hours s at room temperature. The primary antibody was washed off and the cells were incubated with AF-488 Goat anti-mouse secondary antibody for 1 hour. The antibody dilutions and catalog number can be found in Table 2. The sample was washed with PBS and the chamber attachment was removed from the slide and mounted with DAPI DABCO mounting medium (90% Glycerol, 25 mg/mL DABCO, 2.5 ng/mL DAPI in 1×PBS, pH 8.6) mounting medium and sealed with a coverslip. The slides were imaged using a 40×oil immersion lens on the FITC and DAPI channels. Five fields of view were imaged in each well, for each condition three wells were imaged. Data was collected in this fashion for three replicates for each of the M4C8, F7C6, and CCND2-hiPSC lines. The collected data was de-identified and given to a researcher for blinded analysis of CM a) sarcomere length using SarcOptiM ImageJ plugin94 b) cell perimeter c) cell area d) aspect ratio e) nucleation. The aspect ratio was defined as the longest axis of the cell that intersects the nucleus (the long axis) divided by the longest distance that is orthogonal to the long axis and intersects the nucleus (the short axis).

### Immunocytochemistry and Immunohistochemistry of Direct Co-Cultures and EHTS

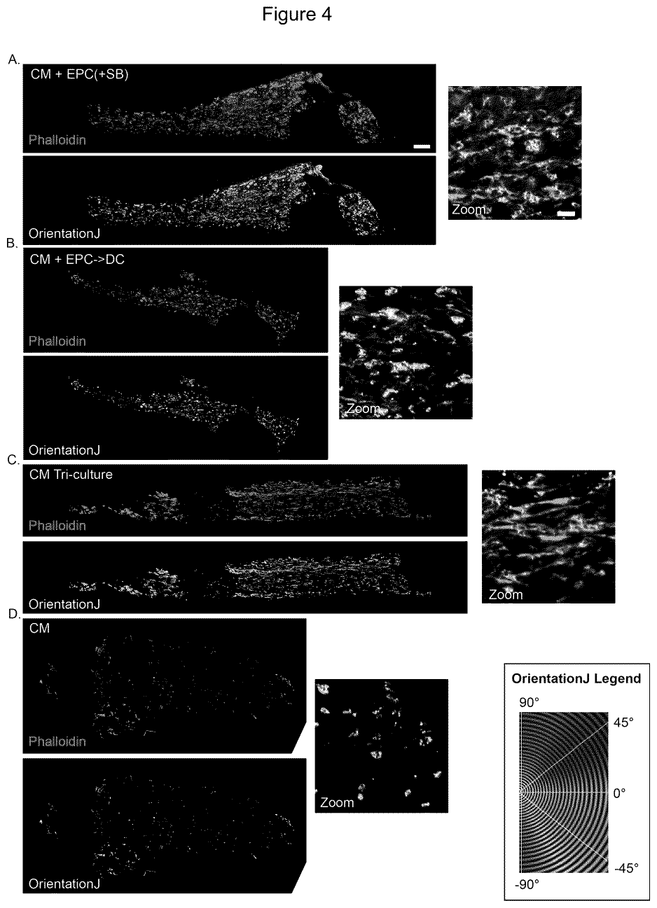

2D co-cultures seeded in 48-well plates and EHTs were preserved in 4% paraformaldehyde in PBS with 90 mM KCl, 4 mM EDTA, and 4 mM MgCl2 for diastolic arrest of the CM⁹⁵. Briefly, the cells or EHTs were washed with DPBS without calcium or magnesium and then incubated for either 15 minutes (2D) or 45 minutes (EHTs) at room temperature followed by washing with PBS twice before storing in PBS with 0.1% sodium azide at 4 °C until staining (2D) or cryoembedding (EHTs). To process EHTs for cryo-embedding the fixed tissue was dehydrated in 30% sucrose for 48 hours. After dehydration the EHTs were cut in half and transferred into 50:50 30% Sucrose: OCT for 30 minutes then transferred into cryo-molds containing OCT and frozen in the −80 °C until cryo-sectioning. Using a cryostat (Leica CM1900) the EHTs were sectioned across the center to determine the cross-sectional area as well as transversely at a 5-10 μm thickness. For staining, both the 2D wells and EHT sections were permeabilized using 0.2% Triton-X-100 for 1 hour and blocked in BGST for 3 hours before an overnight incubation at 4 °C with the primary antibody dilutions in BGST. After the overnight incubation, the samples were washed twice with 0.2% Tween-20 and then twice with PBS. The samples were then incubated with secondary antibody dilution in BGST for 1.5 hours and then washed twice with 0.2% Tween-20 and with PBS. The samples were incubated with 1 g/L DAPI in PBS for 20 minutes and washed with PBS 3 times. The EHT sections were mounted in Prolong™Glass Antifade Mountant (Thermo P36982). The 2D wells were imaged at 20× magnification on a fluorescent microscope (Leica) and the EHT sections were imaged on an Olympus FluoView IX2 Inverted Confocal microscope with a 60×oil immersion lens. Phalloidin and DAPI images for tissue alignment as well as brightfield and phalloidin images for EHT cross-sectional area measurements were imaged at 20× magnification on a fluorescent microscope (Leica) using the ImageJ OrientationJ plugin (FIG. 4). The antibody dilutions and catalog number used for IHC can be found in Table 2.

### Transmission Electron Microscopy of Co-Cultured CM

The 2D co-culture of conditions CM+EPC(+SB) and CM (+SB) were seeded as described above on 13 mm Thermonox® (Nalge Nunc International) plastic coverslips coated with 17.36 μg/cm² Cultrex-RGF and cultured for 9 days. The cells on the coverslips were fixed in 3% paraformaldehyde and 1.5% glutaraldehyde in 0.1M sodium cacodylate buffer with 2.5% sucrose, 5 mM calcium chloride, and 5 mM magnesium chloride (pH 7.4) for 1-2 hr at room temperature (RT) after 8-days of co-culture. Cell cultures were rinsed in buffer (3×, 5 min ea), fixed in 1% OsO4 in 0.1M sodium cacodylate buffer for 1 hr at RT, rinsed in ultrapure water (NANOpure InfinityR; Barnstead/Thermo Fisher Scientific; Waltham, Maryland) (3×, 5 min ea), en bloc stained with 1% aqueous uranyl acetate for 1 hour at RT, and rinsed in ultrapure water (3×, 5 min ea). Samples were then dehydrated in an ethanol series (50%, 75%, 95% (all 2×), 100% (3×), 5 min ea) and infiltrated with Embed 812 resin (Electron Microscopy Sciences, Hatfield, Pennsylvania) (1:1 ethanol: resin, 2 hrs, 100% resin with catalyst, 2 hrs, 2×). Cultures were embedded by placing the coverslips upside-down on top of resin-filled capsule molds, then polymerized in a 60C oven for 48 hours. Ultrathin sections 80-100 nm thick were cut on a Leica Ultracut UCT microtome using a diamond knife and collected on formvar/carbon-coated copper slot (2 mm×1 mm) grids (Electron Microscopy Sciences, Hatfield, Pennsylvania). They were stained with 3% aqueous uranyl acetate for 20 min, rinsed in ultrapure water (15 sec, 5×), stained with Sato's triple-lead stain (Sato 1968) for 3 min, and rinsed in ultrapure water (15 sec, 5×). Sections were examined with a JEOL JEM1400-Plus transmission electron microscope operating at 60 kV. Images were recorded with an Advanced Microscopy Techniques XR16 camera using AMT Capture Engine software ver. 7.0.0.187.

### Single-Cell RNA Sequencing Sample Preparation

Single-cell RNA sequencing was done following the supplier's manual for the Chromium Next GEM Single Cell Sequencing Kits. The 2D co-cultures were singularized using 0.25% trypsin-EDTA for 20 minutes at 37 °C as described above. The viability of the cells was determined via propidium iodide on the BD Accuri C6 flow analyzer and then the cells were prepped for fixed single-cell sequencing according to the Chromium Next GEM Single Cell Fixed RNA Sample Preparation Kit (10× Genomics 1000414). Libraries for single-cell sequencing were created using the Chromium Fixed RNA Kit, Human Transcriptome, 4 rxns×4 BC (10× Genomics 1000475) by following the manual for probe hybridization and library creation. The samples were sequenced according to the 10×genomics FLEX single-cell sequencing workflow at the University of Minnesota Genomics Center (UMGC). Briefly, pooled libraries were denatured and diluted to the appropriate clustering concentration. The libraries were then loaded onto the NovaSeq paired end flow cell and clustering occurred on board the instrument. Once clustering was completed, the sequencing reaction immediately began using the Illumina 2-color SBS chemistry. Upon completion of read 1, 2 separate 8 or 10 base pair index reads were performed. Finally, the clustered library fragments were re-synthesized in the reverse direction thus producing the template for paired end read 2.

Single-cell RNA sequencing analysis Reads were aligned to the human reference genome provided by 10× Genomics (refdata-gex-GRCh38-2020-A) using cellRanger (v 7.1.0) and the Chromium_Human_Transcriptome_Probe_Set_v1.0_GRCh38-2020-A. Analysis was performed using R (v 4.1.0). Libraries were quality-controlled individually as follows: 1) The matrix of filtered counts per cell barcode was analyzed using Seurat (v 4.3.0.1). 2) Empty droplets were filtered out with a minimum total RNA and Feature counts per barcode based on their count distribution on a per-sample basis. 3) The presence of multiplets was assessed using the scDblFinder (v 1.8.0) package, followed by manual filtering of multiplets of the same cell type using an upper RNA count limit. 4) Gene expression was estimated on the clean data using the transformed counts (SCTransform) with the percent of mitochondrial reads as covariate. To take full advantage of replication while avoiding confounding batch effects, biological replicates of the same condition were integrated into a single object using Seurat's standard workflow. Then, integrated treatment objects were merged into a single object and renormalized (SCTransform). This combined integration/modeling approach yielded cell type proportions that correlated with the expected experimental outcomes based on the seeded cell types, while the default integration or covariate batch modeling only did not. Principal component (PC) analysis of the combined object was performed for dimensionality reduction. UMAP reductions were used only for visualization purposes. Cell classification was performed using the FindNeighbors and FindClusters functions (Seurat) based on the first 25 PCs and the normalized counts. Cell cluster identification was performed based on their relative expression of genes of interest for the CM and EPC populations, their marker genes, and cluster abundance per treatment. Differential gene expression tests were done with the default parameters (test.use=“wilcox”). Overrepresentation Analysis (ORA) was performed on all sets of differentially expressed genes (DEGs) using the clusterProfiler (v 4.2.2) package and the gene ontology and KEGG databases in the msigdbr (v 7.5.1) package. Term and gene set databases were filtered to contain only genes detected across all treatments in the experiment (14263 genes). In detail, DEGs for each identity class were separated into up or down-regulated lists, the maximum between all or the top 300 of each list, ranked by the average log 2 fold change, was tested with the enricher function using p-value and q-value cutoffs of 0.05 and the Benjamini-Hochberg correction for multiple hypothesis testing. Each of the three subcategories of the GO terms (BP, MF, and CC) and the KEGG database were tested separately. Further details concerning the software used are in Table 3.

qPCR for Maturation Markers

The EHTs from different groups were lysed using 600 ul of RLT-lysis buffer containing β-mercaptoethanol (RNeasy kit (Qiagen)). Subsequently, the lysate was further homogenized using 21G and 25G needles (~10 times each) for complete cell lysis. Total RNA was isolated using the RNeasy kit according to the manufacturer's protocol, followed by on-column DNA digestion using DNasel to remove any traces of DNA as per the instructions. The elution was performed using pre-heated (80C) DNase/RNase-free water. To obtain a higher yield, we perform a second round of elution using the eluate. The quantity and quality were assessed by measuring absorbance at 260 nm and 280 nm using a microplate reader instrument. For cDNA synthesis, 250 ng of total RNA was used and synthesized using the SuperScript IV VILO kit (Thermo Fisher Scientific) according to the manufacturer's protocol in a 20 ul reaction volume. The reaction mix was diluted 10 times using DNase/RNase-free water for the next steps. Quantitative PCR (qPCR) was performed using gene-specific oligos and the SYBR-green method. The list of oligos used is provided in Table 4. For the qPCR analysis, each transcript was normalized to the housekeeping gene, EDF1. To visualize CM-specific changes, we further normalized the transcript levels to the levels of TNNT2.

### Statistics

All statistics were done in PRISM using a one-way or two-way ANOVA and post-hoc Fisher LSD comparison of means. Significance was indicated as follows ****p≤0.0001, **p≤0.001, **p≤0.01, *p≤0.05.

### Results

### Mouse Embryonic CM Maturation and Proliferation at E10, E12, and E17

The degree of CM maturation that occurs during embryonic development is understudied. To explore this and the potential role of the epicardium in embryonic CM maturation, three embryonic time-points were identified that correspond to well-known dynamics of the formation of the epicardium but limited knowledge of CM maturation at the cellular scale. E10 was chosen as it corresponds to a time-point where the primary heart tube is spontaneously contracting and the proepicardial organ is fully formed but few epicardial cells have migrated to cover the heart tube. E12 corresponds to a time-point in murine development where epicardial cells have fully encapsulated the heart to form the epicardium but substantial amounts of EMT have yet to begin. E17 corresponds to a time-point in mouse development where substantial amounts of EMT have occurred and stromal cells are present throughout the myocardium.

During embryonic development, CM remain proliferative but almost entirely exit the cell cycle in the first week after birth. Thus, decreases in proliferation are a sign of advancing maturation in CM. CM proliferation was examined in parallel with common hiPSC-CM maturation parameters to see if the maturation indices 1) happened during embryonic development and 2) showed the same proliferation-maturation dichotomy as has been demonstrated in postnatal development. Sarcomere length was assessed using staining for aActinin (FIG. 2A). CM area and perimeter was assessed via wheat-germ agglutinin (WGA) for membrane visualization, nkx2.5 to mark CM, and DAPI staining (FIG. 2B). Lastly, the percent of proliferating CM were visualized and quantified using the colocalization nkx2.5 with Ki67 and DAPI (FIG. 2C). The sarcomere length increased substantially over these developmental time points from 1.44±0.11 μm at E10 to 1.59±0.23 μm at E12 and 1.73±0.20 μm at E17 (FIG. 2D). CM area and perimeter showed increases as well (FIG. 2E-F). CM proliferation at E10 was the greatest (75±3%) and decreased by ~10% at E12 (63±10%) and another 10% at E17 (54±6%) (FIG. 2G). These findings confirm that even at an early time point, when CM proliferation is high, CM proliferation is inversely related to maturation in vivo. They also confirm that substantial amounts of CM maturation occur during embryonic development thus hiPSC-CM can potentially be driven to mature by mimicking embryonic interactions with epicardial cells.

### Epicardial Co-Culture Composition in 2D

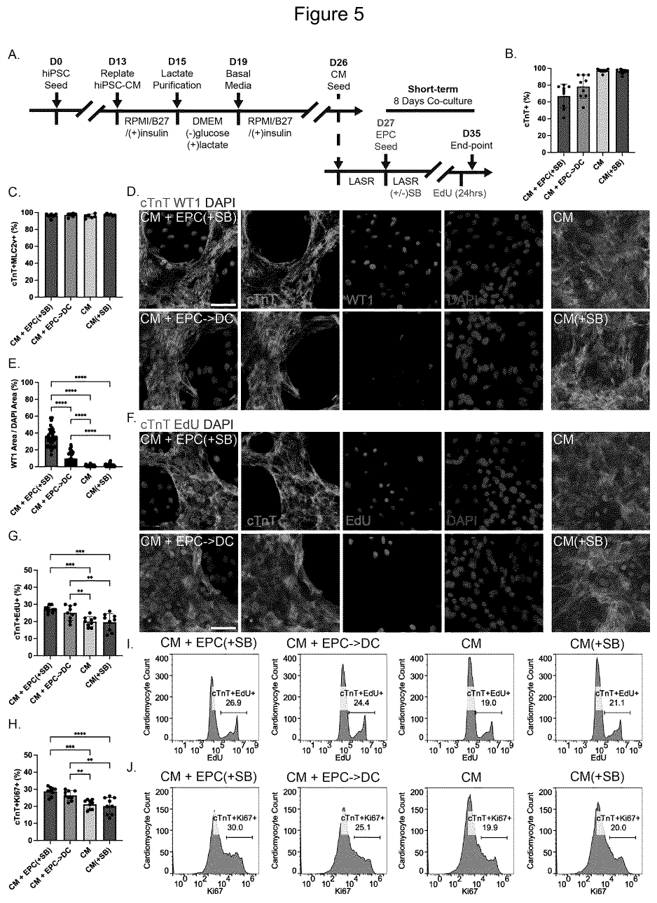

The effect of epicardial cells on hiPSC-CM maturation and function was first explored in 2D. This allowed for the effect of epicardial cells on individual CM cellular maturation to be determined without confounding the effects of tissue architecture and compaction in 3D. Epicardial cells were seeded on top of CM at a 1:3 ratio and the co-cultures were either maintained in TGF-b inhibitor (SB431542) to prevent EMT (CM+EPC(+SB)) or in basal epicardial media to allow spontaneous EMT into epicardial derived cells (CM+EPC→DC) to occur (FIG. 5A). The CM+EPC→DC condition allowed us to study a situation in which the epicardial cell population is gradually being diluted by the emergence of stromal cells, as in development. Extended exposure to ascorbic acid-containing media was used to drive a ventricular phenotype of the hiPSC-CM in all conditions to limit variation between other CM subtypes. Flow cytometry was used to assess the percentage of CM via cardiac troponin T (cTnT) staining and the proportion of ventricular CM was determined via co-staining of cTnT and myosin light chain 2v (MLC2v). Due to the expansion of the epicardial population in the presence of the TGF-b inhibitor, the CM+EPC(+SB) conditions contained 67±14% cTnT positive CM and the CM+EPC→DC condition contained 78±14% after 8 days of co-culture (FIG. 5B). The CM population in the CM controls without (97±2%) and with (96±3%) TGF-b inhibition remained high (FIG. 5B). Epicardial co-culture did not increase the percentage of ventricular CM in culture since all conditions contained 97% MLC2v+CM (FIG. 5C).

Immunocytochemistry (ICC) for cTnT, Wilms tumor 1 (WT1), an epicardial marker, and DAPI were used to assess the percentage of epicardial cells remaining after 8 days of co-culture (FIG. 2D). Of note, there was substantial migration of epicardial cells into the CM layer depicted by red nuclei interspersed within the cTnT positive area (FIG. 2D). Additionally, large patches of highly confluent epithelial cells can be seen separate from the CM in bright field images (Figure S1A). Quantification of the WT1 cell population determined that the epicardial co-culture maintained in TGF-b inhibitor contained 35±11% WT1 positive cells while a significant portion of the epicardial cells in the EPC→DC conditions underwent EMT and only 10±7% WT1 positive cells remained (FIG. 2E). Three hiPSC-lines were used for these experiments and the maintenance of WT1 positive cells as well as the cTnT positive population is consistent between all three lines in each condition (Figure S1B-D). The MLC2v+CM population was consistent across the two lines tested (Figure S1E-G). These data serve to validate the composition of cells present in the 2D in vitro model.

### Epicardial Cells Enhance CM Proliferation in 2D

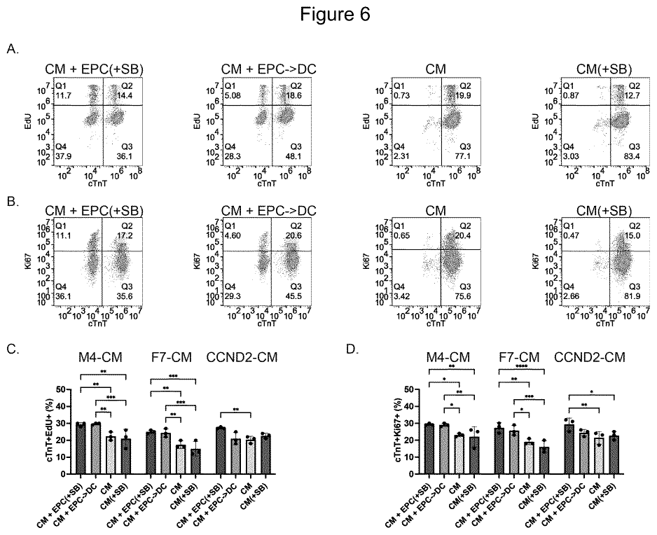

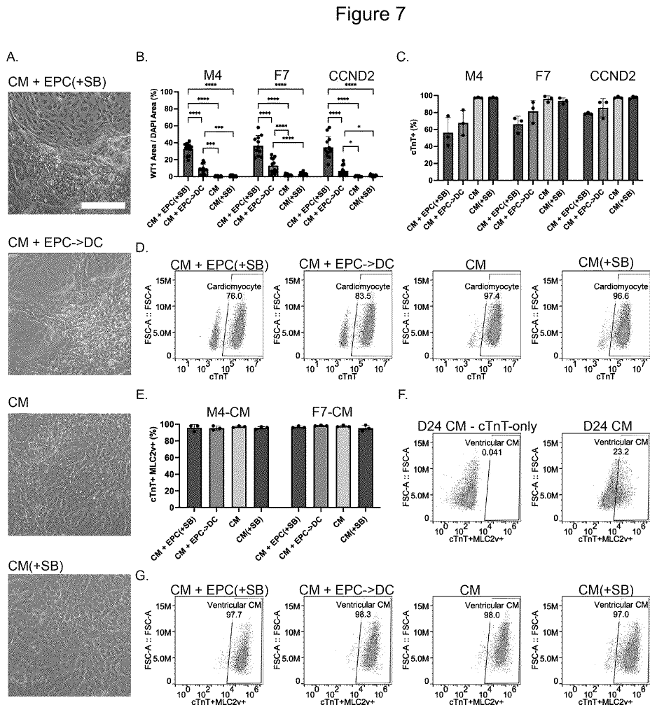

Since multiple animal studies show that epicardial cells are essential for CM proliferation during development we aimed to explore the ability of hiPSC-derived epicardial cells to enhance hiPSC-CM proliferation. The co-cultures and controls were given the thymidine analog Ethynyl deoxy-Uridine (EdU) for the last 24 hours in culture and stained for both cTnT and incorporated EdU or Ki67 a nuclear transcription factor that marks cell proliferation (FIG. 6A-B). The percent proliferation as indicated by the cTnT+EdU+ population increased from ~20%, in both CM and CM (+SB) conditions, to 27±2% percent in the EPC(+SB) conditions and 25±4% in the EPC→DC condition (FIG. 5F-G). This increase in proliferation was consistent with the expression of the nuclear proliferation marker Ki67 (FIG. 5H). Histograms of the CM population versus EdU or Ki67 were used to determine the percentage of proliferative CM (FIG. 5I-J). This confirms that hiPSC-derived epicardial cells, that are maintained or are currently undergoing EMT, both increase CM proliferation as in animal models during development. This finding was consistent for the CM+EPC(+SB) condition in all three hiPSClines (FIG. 6C-D). It was also consistent for the CM+EPC→DC condition for the M4 and F7 lines but not the CCND2-hiPSC line. We noted that the co-cultures from the CCND2 line exhibited a lower percentage of EPCs in the CM+EPC→DC condition (FIG. 7B). This could reflect a more robust EMT of the CCND2-EPCs, that the EPCs themselves are less proliferative, or since this line is genetically modified to increase CM proliferation, the initial proliferation rate of the CM could have outcompeted the EPCs resulting in the higher cTnT+ populations values for both the CM+EPC(+SB) and CM+EPC→DC conditions (FIG. 7C). In summary, EPCs spur proliferation, but a large enough dose may be required to do so, and that dose might be dependent on the characteristics of the stem cell source line.

### Epicardial Cells Enhance Select Indices of CM Functional Maturation in 2D

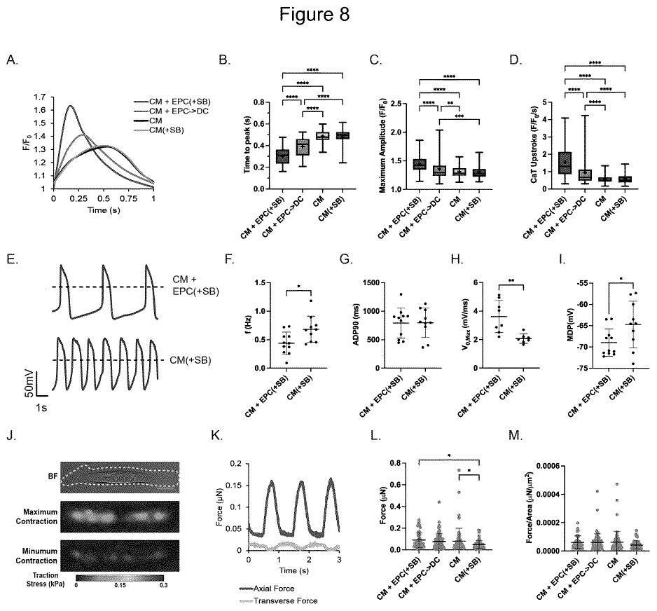

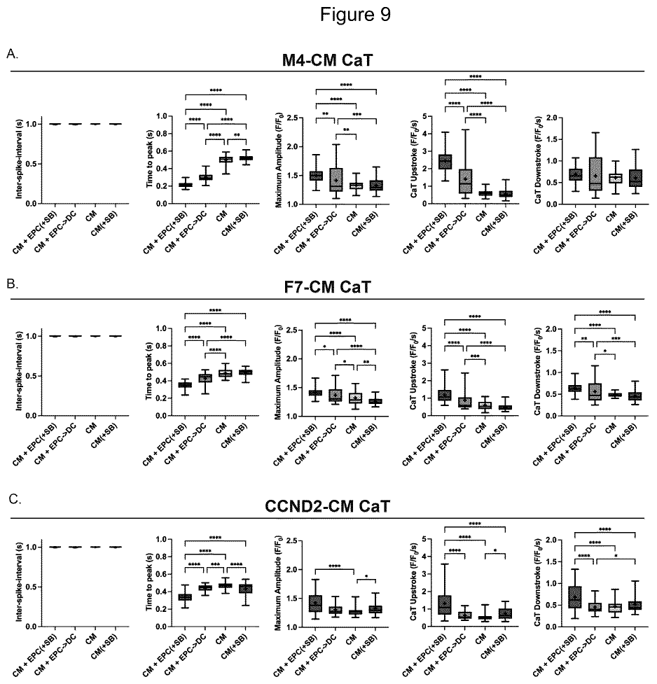

To determine whether and to what extent functional maturation of CM in the epicardial and epicardial-derived cell co-cultures occurred, calcium transient (CaT) assessment was conducted at the experimental endpoint. From embryonic to post-natal and adult development there are stark increases in the amount and rate of release and sequestration of calcium from the sarcoplasmic reticulum as calcium is the key mediator of excitation-contraction coupling. The CM co-cultures were incubated with calcium-sensitive dye and imaged with 1 Hz electrical stimulation, such that the change in fluorescent intensity with time represents the calcium handling of the CM (FIG. 8A). Most notably, the presence of epicardial cells resulted in a stark decrease in CaT time to peak from 0.48 s in both CM control conditions to 0.30 s in the EPC(+SB) condition and 0.39 s in the EPC→DC condition (FIG. 8B). The robust decrease in time to peak for the epicardial co-culture conditions was consistent across all three hiPSC-lines (FIG. 9). Overall, the CM+EPC(+SB) condition showed the most mature calcium handling dynamics followed by the CM+EPC→DC condition. Both conditions showed increased maximum amplitude and CaT upstroke velocity (FIG. 8C-D). This increase was seen across all three hiPSC-lines in the CM+EPC(+SB) condition but was lacking in the CCND2 CM+EPC→DC condition most likely due to the lowest remaining amount of epicardial cells in this line (FIG. 9). There was also a significant increase in downstroke velocity for the CM+EPC(+SB) condition on average and in two of the hiPSC-lines (FIG. 9).

To further assess the electrophysiologic properties of CM in the CM+EPC(+SB) condition and its control, CM (+SB), the cells were replated at a low density, and patch clamp for CM action potential (AP) was performed (FIG. 8E). Maturation of ventricular CM is characterized by rapid membrane depolarization, slight rapid repolarization to create the “notch” seen at the peak of the AP, an extended plateau phase, and then repolarization back to the resting membrane potential⁵³. The APs of hiPSC-CM have slower upstroke velocities during the rapid depolarization phase, lack of a “notch,” and shorter plateau phases due to immature ion channel compositions. The CM from the epicardial co-culture had a significant decrease in beat rate frequency (f) (FIG. 8F). The CM (+SB) and CM+EPC(+SB) both have a long AP duration at 90% repolarization (ADP90) of ~800 ms which is characteristic of ventricular CM (FIG. 8G). Consistent with the CaT data, the maximum upstroke velocity was almost doubled in the CM+EPC(+SB) (FIG. 8H). Additionally, a decrease in maximum diastolic potential from −65±6 mV to −69±3 mV was seen in the epicardial co-cultures suggesting a trend toward the resting potential of mature adult ventricular CM of −85 mV2. In summary, patch clamp data further support increased electrochemical maturation of CM in epicardial co-cultures.

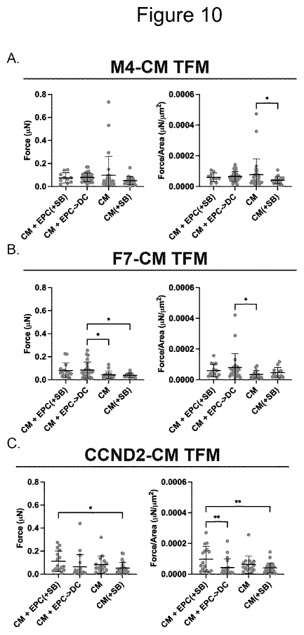

Another functional attribute of maturation is increased force generation at the tissue (ventricle) and cellular (CM) levels⁵. To explore the cellular level mechanical function of the hiPSC-CM the co-cultures were singularized and plated onto micropatterned substrates of a known Young's Modulus that contained fluorescent beads. The displacement of the beads from the CM contractions was used to calculate the force generated by each CM; this method is known as traction force microscopy (TFM) (FIG. 8J-K). There were no significant differences between the CM-only conditions and the epicardial co-culture conditions. There was however a significant decrease in force in the CM (+SB) condition compared to the CM condition (FIG. 8L). The negative effect of the TGF-b inhibitor was ameliorated by the positive effect of the epicardial cells; there was a significant increase in the CM force in the CM+EPC(+SB) condition compared to the CM (+SB) condition (FIG. 8L). However, when normalized to the CM area there are no significant differences in CM stress generation (FIG. 89M). The force generation data is broken down by line in the supplement (FIG. 10). These data indicate that the effect of epicardial cells on CM maturation is more substantial with regard to electrochemical parameters and does not appear to include mechanical maturation at a cellular level.

### Epicardial Cells Enhance CM Morphologic Maturation in 2D

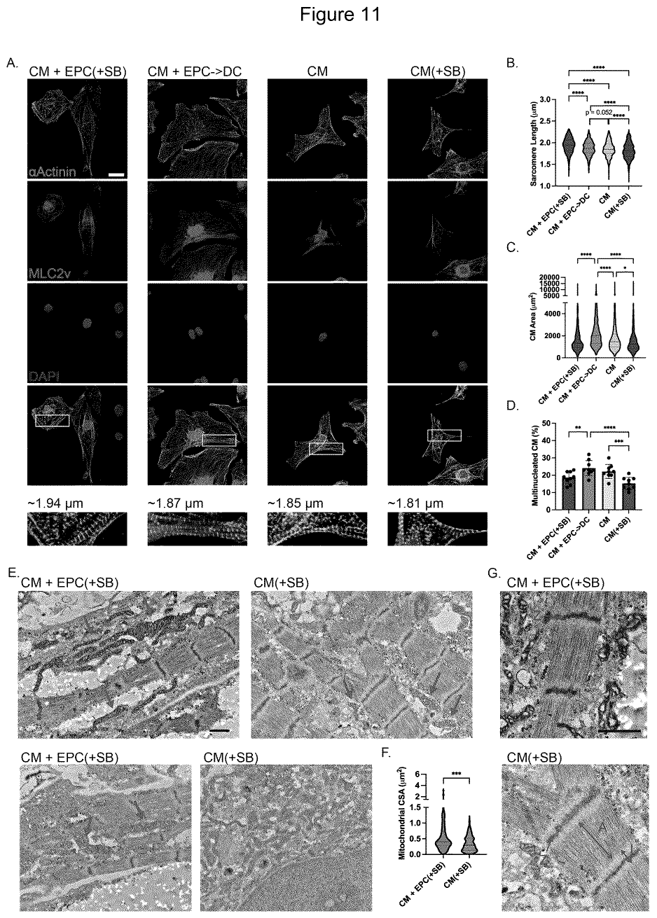

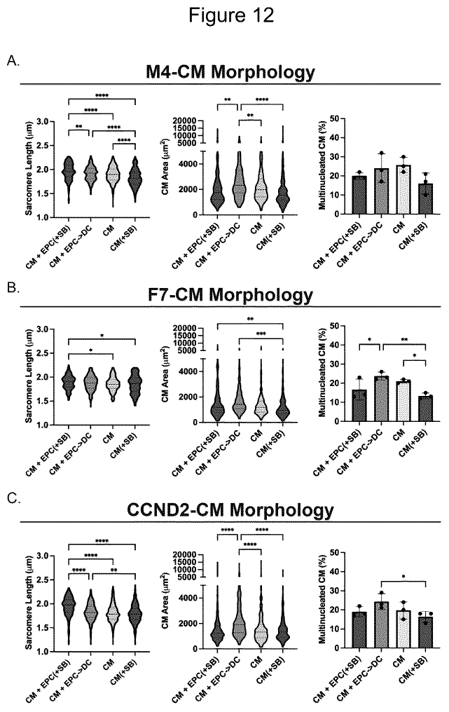

We next evaluated the CM of EPC(+SB) and EPC→DC co-cultures for morphologic features of maturation. CM from the 2D co-cultures were replated onto glass chamber slides at a low density and fixed in diastole 24 hours later. The sarcomeres were visualized via staining for aActinin and the ventricular phenotype was validated with MLC2v staining (FIG. 11A). The adult myocardium has an average sarcomere length of 2.25 μm when arrested in diastole56. The longest sarcomere length was detected in the CM+EPC(+SB) co-cultures at 1.94 ±0.17 μm followed by 1.87±0.16 μm in the CM+EPC→DC condition while the CM condition had an average length of 1.85±0.17 μm (FIG. 11B). Without the aid of epicardial cells, the TGF-b inhibition resulted in a significant decrease in CM (+SB) sarcomere length (1.81±0.18 μm). The increase in CM sarcomere length with epicardial cell co-culture was consistent across all three hiPSC-lines whereas TGF-b inhibition only negatively affected sarcomere length in the M4-hiPSC line (FIG. 12).

Additionally, the epicardial-derived co-culture resulted in CM hypertrophy, an indicator of maturation, as seen by a significant increase in CM area that was not observed in the other conditions (FIG. 11C). This could indicate a specific role of epicardial-derived cells on CM hypertrophy not afforded by the epicardial cells. Since TGF-b has been linked to hypertrophy it is possible that though the CM are still maturing in the CM+EPC(+SB) conditions, hypertrophy is inhibited by the TGF-b inhibitor57-59. The increased CM area in the CM+EPC→DC condition is consistent across all three hiPSC-lines (FIG. 12). Another morphologic indicator of maturation is the percent of multinucleation in hiPSC-CM as the adult human heart contains ~26% multinucleated CM44. No significant increases in multinucleation were seen in the epicardial co-cultures (FIG. 11D). This could be due to the already high percent of ~20% multinucleated CM in all conditions. Though, TGF-b inhibition caused a significant reduction in the percentage of multinucleated CM in the CM (+SB) condition; the presence of epicardial cells ameliorated this negative effect. The results were similar in all three hiPSC-lines (FIG. 12). In summary, in the CM+EPC(+SB) condition, CM showed increases in sarcomere length while in the CM+EPC→DC condition the CM showed increased hypertrophy; this outcome indicates differential effects of EPCs and EPDCs on hiPSC-CM phenotype though the functional data was similar.

Since the most significant maturation effects were seen in the CM+EPC(+SB) condition, transmission electron microscopy (TEM) was conducted on the CM+EPC(+SB) conditions and its control, CM (+SB), to better visualize CM ultrastructure as well as to quantify the size of mitochondria present. The mitochondria of CM+EPC(+SB) tended to be in direct apposition to sarcomeres, a characteristic of maturing CM, whereas the mitochondria of CM (+SB) were present most frequently in separate clusters (FIG. 12E). The CM+EPC(+SB) condition had a significant increase in mitochondrial cross-sectional area in comparison to the CM (+SB) control (FIG. 12F). Mature adult-CM sarcomere TEM images contain the distinct presence of Z-lines, I-bands, A-bands, and M-lines12. Both conditions have the distinct presence of Z-lines, Ibands, and A-bands but lack the presence of M-lines in CM (FIG. 12G). The M-line typically appears last with long-term culture, 3D cultures, and extended exposure to electrical stimulation and does not occur until post-natal development in animal studies and so was not expected here.

### Single Cell RNAseq Reveals a Unique Population of vCM in Both Epicardial Co-Cultures

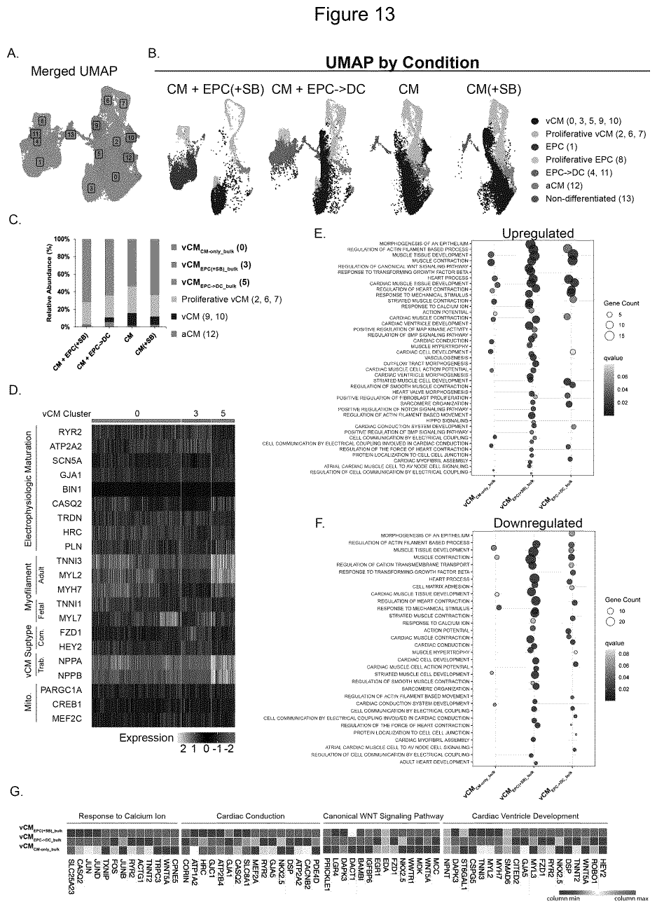

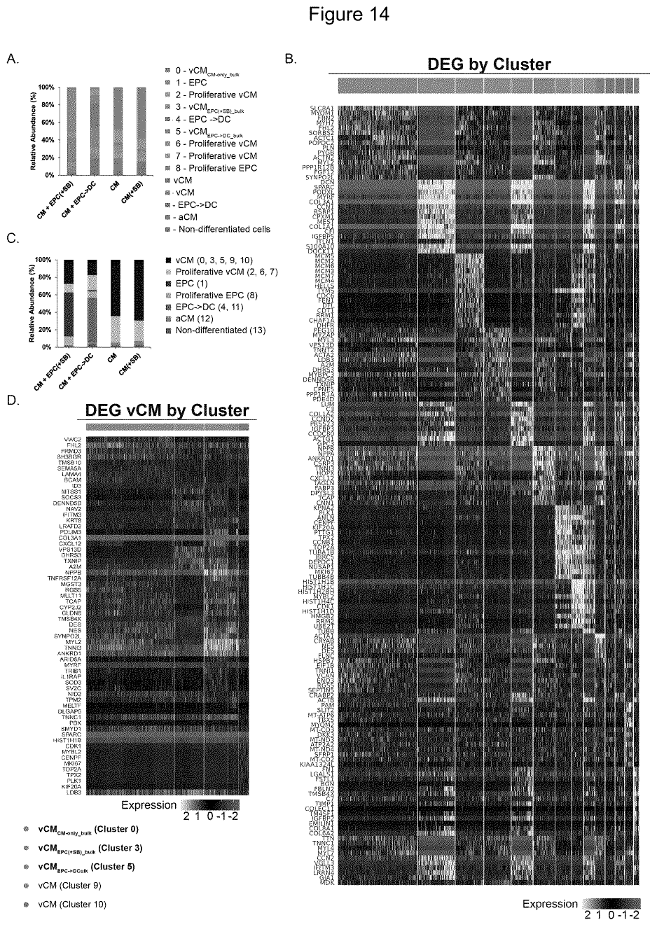

To further understand the genetic landscape of the CM in the epicardial co-cultures single cell RNA sequencing was conducted on all 4 conditions in duplicate. The cells from all the conditions and replicates were merged and 14 clusters were identified (FIG. 13A). The proportions of cells in each cluster by conditions can be visualized in the supplement (FIG. 14A). These were identified as seven cell type categories, the ventricular CM (vCM—Clusters 0, 3, 5, 9 and 10), proliferative vCM (Clusters 2, 6 and 7), EPC(Cluster 1), proliferative EPC(Cluster 8), epicardial to epicardial-derived cells (EPC→DC—Clusters 4 and 11), atrial CM (aCM—Cluster 12) and non-differentiated cells (Cluster 13) that represent the 1-4% cTnTnegative population from the initial hiPSC-differentiation (FIG. 13B). The identity of each cluster was determined by looking at the differentially expressed genes between each cluster and all other cells (FIG. 14B). In particular, the vCM expressed cardiac markers as well as the ventricular-specific myofilament isoform MYL2. The proliferative vCM had similar gene expression as the vCM but also expressed markers related to mitotic cell division, chromosome segregation, and/or cytokinesis. The aCM expressed general cardiac markers, TNNT2, and the atrial marker NPPA but lacked expression of MYL2. The proportion of cell types across conditions shows a large population of EPC in the CM+EPC(+SB) and a genetically distinct population of cells emerging in the CM+EPC→DC conditions labeled EPC→DC (FIG. 14C). To guide analysis, the 9 clusters expressing CM marker genes were grouped into 6 categories: i) vCMCM-only_bulk (Cluster 0), ii) vCMEPC(+SB)_bulk (Cluster 3), iii) vCMEPC→DC_bulk (Cluster 5), iv) Proliferative vCM (Clusters 2, 6 and 7), v) vCM (Clusters 9 and 10) and vi) aCM (Cluster 12) (FIG. 13C). The majority of CM from the CM-only conditions fall into the vCMCM-only_bulk category (cluster 0 CM-50% and CM (+SB)-61%) while 69% and 17% of the CM in the CM+EPC(+SB) and CM+EPC→DC conditions respectively fall into the vCMEPC(+SB)_bulk category (cluster 3). The majority of the CM from the CM+EPC→DC condition falls into the vCMEPC→DC_bulk category (cluster 5-43%) (FIG. 13C). Separate clustering of vCM from the co-cultures indicated significant differences in gene expression stimulated by EPC and EPC→DC in the cocultures.

To further understand these genetic shifts, genes related to CM maturation were examined, between the vCMCM-only_bulk, vCMEPC(+SB)_bulk, and vCMEPC→DC_bulk (Cluster 0, 3 and 5). Largely consistent with functional characterization, the vCMEPC(+SB)_bulk (Cluster 3) showed an increase in the key electrochemical maturation genes ryanodine receptor 2 (RYR2), ATPase sarcoplasmic reticulum calcium transporter 2 (ATP2A2) and triadin (TRDN) (FIG. 13D). Surprisingly, they also showed a marked decrease in other key electrochemical maturation genes, such as calsequestrin 2 (CASQ2), histidine-rich calcium-binding protein (HRC), and (phospholamban) PLN. Additionally, though sarcomere length was increased in the CM+EPC(+SB) co-culture, there was a surprising decrease in the adult ventricular myofilament isoforms cardiac muscle troponin I (TNN13), myosin light chain 2 ventricular form (MYL2) and myosin heavy chain 7 (MYH7). Nonetheless, the vCM were more specified showing increases in the compact ventricular markers frizzled 1 (FZD1) and the related family bHLH transcription factor with YRPW motif 2 (HEY2) along with decreases in trabecular ventricular myocardium markers natriuretic peptide A (NPPA) and natriuretic peptide B (NPPB). Conversely, the vCMEPC→DC_bulk (cluster 5) showed a distinct increase in the adult myofilament isoforms TNN13 and MYL2 with an accompanying decrease in the fetal isoforms skeletal muscle troponin I (TNNI1) lacking in the vCMCM-only_bulk; this fits with the CM hypertrophy seen in the morphological assessment. Interestingly, the vCMEPC→DC_bulk (cluster 5) also showed high expression of the trabecular myocardial markers NPPA and NPPB. These shifts corresponded to an increase in the electrophysiologic maturation marker CASQ2 and PLN but decreases in RYR2 and ATP2A2. These results indicate differential enhancement of electrophysiologic and myofilaments maturation in vCM spurred by either EPCs or EPDCs.

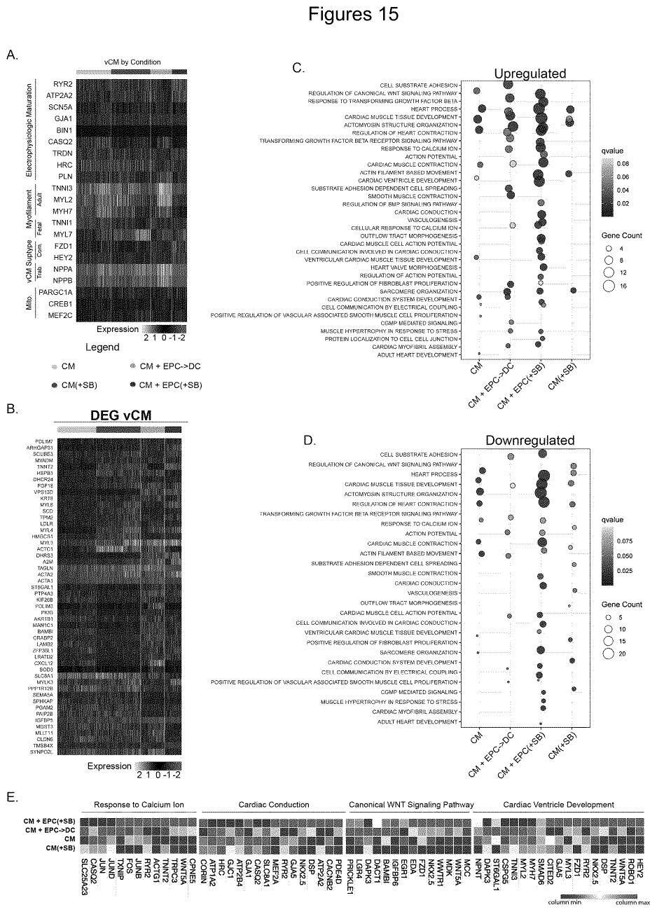

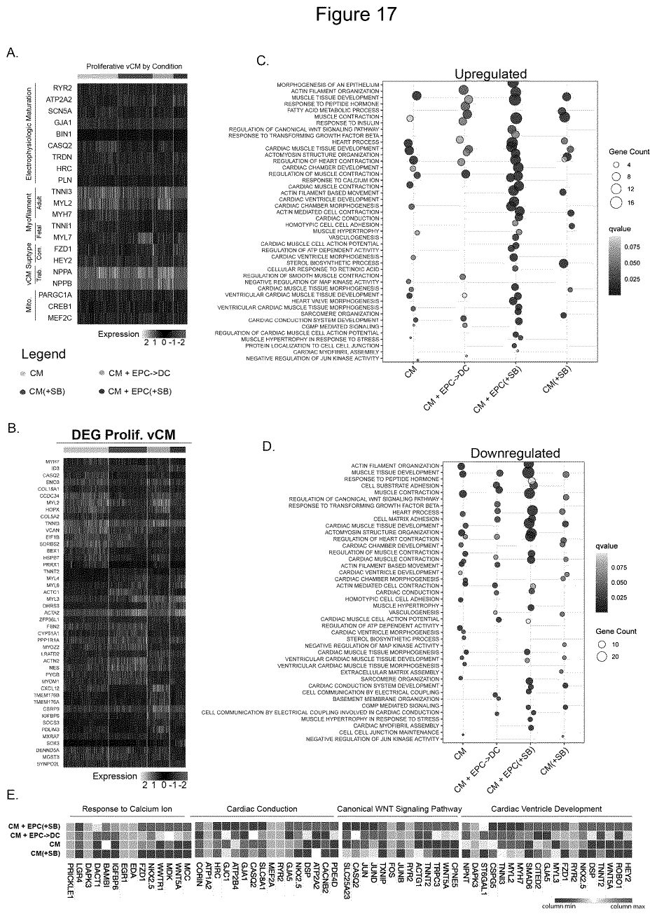

The vCMCM-only_bulk did not have a defined up or downregulation of compact or trabecular markers consistent with early fetal development before ventricular patterning is spurred by the endocardium (trabecular) and epicardium (compact). The same heat map was made for all the vCM pooled by condition (FIG. 15A). In this iteration, the trends are the same as described above but the CM+EPC→DC condition has a portion of cells resembling the vCMEPC(+SB)_bulk in addition to the vCMEPC→DC_bulk. This outcome is consistent with the composition of the CM+EPC→DC condition wherein most, but not all, EPCs undergo EMT. The proliferative vCM show a similar heat map but with muted expression patterns due to the downregulation of cardiac muscle genes during the proliferation process (FIG. 17A).

### Differentially Expressed Genes and Pathways Analysis of vCM in Epicardial Co-Cultures

Differentially expressed genes (DEGs) were determined between each vCM cluster (Clusters 0, 3, 5, 9, and 10) (FIG. 14D). The largest number of DEGs was present in the vCMEPC(+SB)_bulk (Cluster 3), followed by the vCMEPC→DC_bulk (Cluster 5, Table 1). Over-representation analysis (ORA) of gene ontology terms related to biological processes was performed, resulting in many pathways up and downregulated between the different vCM clusters (Table 1). For the vCMEPC(+SB)_bulk multiple pathways related to calcium ion handling, cardiac conduction, and ventricular development were upregulated (FIG. 13E). Conversely, some of these pathways were also downregulated, particularly those related to heart contraction due to decreases in myofilament genes MYL2 and TNNI3 (FIG. 13F). Additionally, pathways related to key signaling cascades that occur during heart embryogenesis were upregulated in the vCMEPC(+SB)_bulk such as Wnt, bone morphogenic protein (BMP), NOTCH, and mitogenactivated protein kinases (MAPK) and HIPPO signaling pathways. These signaling pathways are all highly interconnected and multiple of the same DEGs occur in each pathway. Of particular interest are the WNT signaling pathways because of the role they play in CM proliferation and specification of the compact myocardium in vivo and in vitro^{70, 72-76}. The vCMEPC→DC_bulk showed significant increases in pathways related to myofibrils and muscle contraction but downregulation in some conduction-related pathways (FIG. 13E-F). The vCMCM-only_bulk had the fewest upregulated pathways and among them were a few pathways related to conduction due to increased expression of GJA1, SLC8A1, and HRC (FIG. 13E-F). A heat map showing specific genes that were up and downregulated in four of the enriched pathways shows some of the key differentially expressed genes that contributed to the enrichment (FIG. 13G). Differentially expressed genes and pathways enrichment was also done for the vCM by condition (FIG. 15B-E and Table 1). The same pathways of interest were identified in this analysis. When the proliferative vCM were analyzed by condition there were fewer DEGs and BP pathways that were enriched (FIG. 17B-E and Table 1). However, the ones that were statistically significant confirm results seen when the vCM are analyzed by cluster and by condition.

Overall, these results indicate a significant genetic shift in vCM occurs when they are cocultured with EPC or EPC→DCs. EPCs promote a more fetal-like but electrically conductive vCM population which could be driven by the upregulation of the WNT signaling pathway. The EPC→DC co-cultures bring a population of vCM further along the developmental progression where they are electrically conductive (though with gene expression distinct from EPC cocultures), beginning to express adult myofilament isoforms, and with a population of both trabecular and compact myocardium-like vCM. Next, we moved onto a 3D EHT model to study the effects of EPCs with and without TGF-b inhibition in a more complex system.

CM Proliferation and Function in EHTs Generated with Epicardial and Epicardial Derived Cells

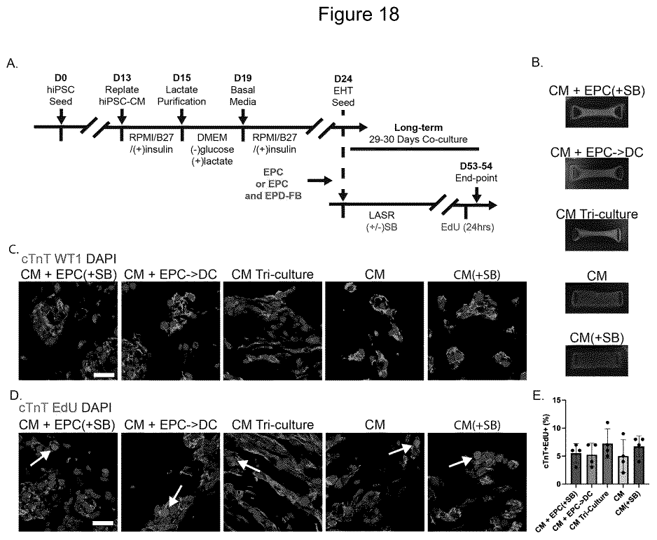

To determine whether the impact of EPCs on CM proliferation and maturation were an artifact of 2D culture or could persist in the context of the more intricate interplay of cell and matrix in a 3D tissue, EHTs were generated with the same four conditions used in 2D and assessed for proliferation and function after 29-30 days of culture (FIG. 18A-B). In addition, a CM Triculture condition was added which included CM, EPCs, and fully differentiated epicardialderived fibroblasts (EPD-FBs) which predominate in the postnatal myocardium. The results for this condition are described in detail later. After 29-30 days of co-culture and similar to 2D, epicardial cells were still present in the CM+EPC(+SB) condition and minimally present in all other conditions (FIG. 18C). EHT proliferation was assessed with cryosections that were contained for cTnT and EdU (FIG. 18D). After 30 days of culture, there were no differences in proliferation between any of the conditions (FIG. 18E). The proliferation of all conditions was substantially decreased from the 2D short-term culture to an average of 5-7% indicative of more advanced maturation of CM in 3D and a transient effect of epicardial cells on CM proliferation that eventually subsides in the context of 3D tissue development.

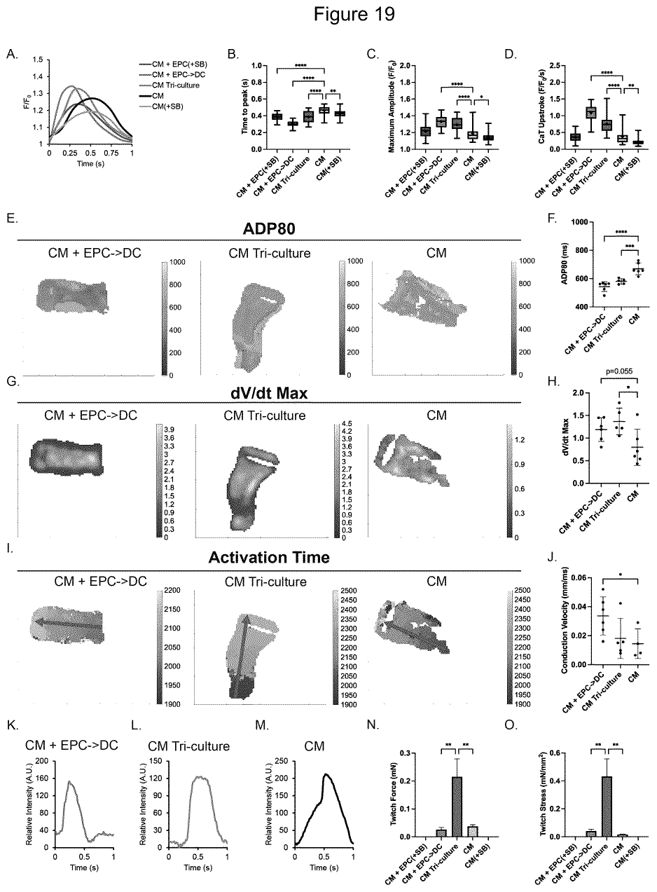

As in 2D, CaT measurements were taken from all five conditions (FIG. 19A). Like 2D, all the co-culture conditions resulted in a robust decrease in time to peak indicating more mature calcium handling (FIG. 19B). In 3D the CM (+SB) condition also resulted in a slight decrease in time to peak but a marked decrease in all other parameters such as maximum amplitude and CaT upstroke velocity (FIG. 19C-D). Differing from the 2D results, the long-term 3D co-cultures required epicardial derived cells (CM+EPC→DC) for a significant increase in maximum amplitude and CaT upstroke velocity (FIG. 19C-D). This could be due in part to long-term exposure to TGF-b inhibitors in the CM+EPC(+SB) conditions. To further explore the role of epicardial and epicardial derived cells on tissue level electrophysiologic maturation optical mapping was conducted for the CM+EPC→DC condition in comparison to a CM-only control. Maps of APD80 showed extended APD80 in the ventricular CM range for all conditions (FIG. 19E). Upon quantification, the longest ADP80 was present in the CM-only control (FIG. 19F). The maximum upstroke velocity (dV/dt Max) was mapped and quantified showing a strong but not significant trend upwards in the CM+EPC→DC condition (p=0.055) (FIG. 19G-H). Lastly, activation time maps were plotted to determine if there was signal propagation from one end of the EHT to another with point electrical stimulation (FIG. 191). If propagation was present, the conduction velocity across the EHT could be calculated. It could be calculated for all 6/6 EHT for the CM+EPC→DC condition but only 4/6 EHTs from the CM-only condition due to inferior tissue connectivity. The conduction velocity showed a significant increase in the CM+EPC→DC condition (FIG. 19J). Representative AP traces show a more regular ventricular AP shape of the co-culture AP and the reduced upstroke present in some of the CM-only Aps (FIG. 19K-M).

Tissue level mechanical function was determined by assessing the twitch force of the EHTs. The long-term incubation with TGF-b inhibitors was detrimental to force generation and both conditions maintained in TGF-b inhibitors for 29-30 days did not have a detectable force (FIG. 19N). Both CM and CM+EPC→DC either had no detectable force or produced small forces in the 0.01-0.02 mN range (FIG. 19N). A similar trend was seen in twitch stress generation (FIG. 19O). Taken together, these data largely mirror those of 2D. While epicardial cells play a role in electrophysiologic maturation in 3D they do not support robust tissue-level force generation.

Addition of an Epicardial-Derived Fibroblast Population in Combination with EPCs Results in Improved Force and Electrophysiologic Function

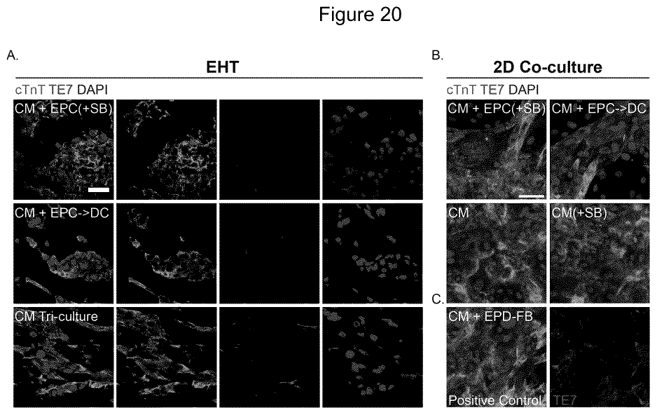

To determine the impact of a more advanced embryonic-to-fetal developmental stage, EPCderived fibroblasts were added to EHTs containing CM and EPCs (CM Tri-culture). In particular, EPCs were maintained in TGF-b inhibitors for 9 days, mimicking the 2D condition, and then the TGF-b inhibitors were removed allowing epicardial EMT to occur with the added EPC-derived fibroblasts (EPD-FB). It was necessary to differentiate the EPD-FB separately as the epicardial cells used in this study lacked TCF21 and did not spontaneously undergo EMT into fibroblasts on their own. The lack of FB in the EPC→DC conditions was validated by staining for TE7, a fibroblast marker, in the 2D and 3D co-cultures (FIG. 20). The addition of fibroblasts did not change CM proliferation in the EHTs (FIG. 18E). When CaT were assessed for the CM Tri-culture, similar improvements were present as in the CM+EPC→DC conditions (FIG. 20A-D). Optical mapping of voltage determined a significant decrease in the average EHT ADP80 as well as a significant increase in the AP dV/dt Max (FIG. 20E-H). All n=5 (from two independent experiments) CM Tri-culture tissues measured did have a measurable conduction velocity (FIG. 201-J). A representative AP trace of the CM Tri-Culture EHTs shows a similar shape to the CM+EPC→DC condition (FIG. 20L). Most significantly, the addition of the fibroblasts to the EHTs resulted in an order-of-magnitude increase in twitch force and stress generation (FIG. 20N-O). Taken together, when the last major cellular component of myocardial development is added to engineered cardiac mimics containing EPCs and EPC>DCs, proliferation is lost, conduction is maintained, and mechanical force generation is significantly enhanced.

### Further Phenotypic Evaluation of EHTs Reveals Enhanced Tissue Level Morphologic Maturation and Restoration of Adult Sarcomeric Isoforms in CM Tri-Culture EHTS

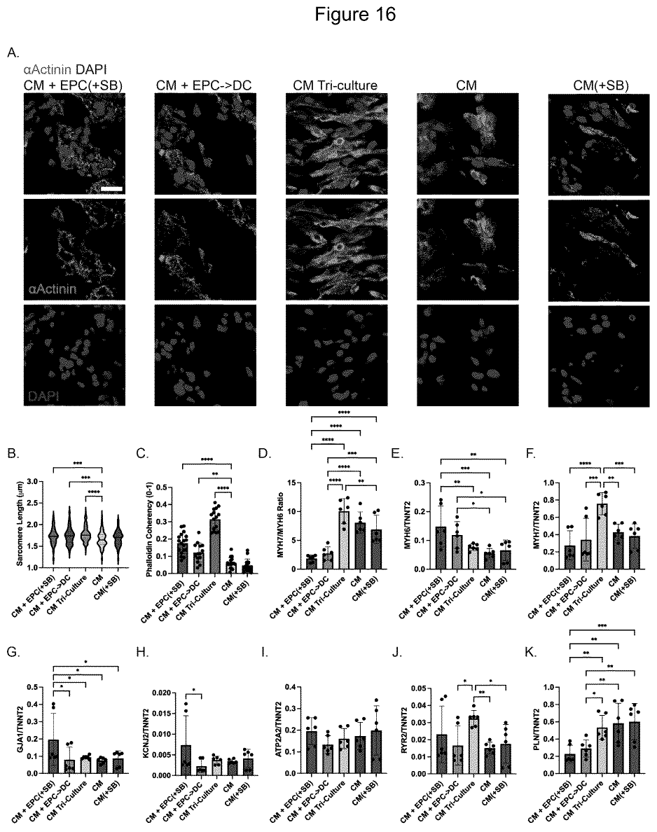

To evaluate CM morphologic maturation in EHTs, tissue cryosections were stained with aActinin and DAPI to assess CM sarcomere length (FIG. 16A). As in 2D, the epicardial cells resulted in a slight but significant increase in sarcomere length that was present in both coculture conditions (FIG. 16B). The CM Tri-culture condition resulted in the largest increase in sarcomere length. Additionally, cellular alignment was evaluated in the EHTs using phalloidin. All co-cultures increased cellular alignment, but the largest increase was present in the CM Triculture: the phalloidin coherency is 0 when cells are randomly aligned and 1 when perfectly aligned (FIG. 16C). To evaluate the level of myofibril adult/fetal isoform switching q-RT-PCR was used to determine the ratio of MYH7 to MYH6 expression (FIG. 16D). We found a significant decrease in the MYH7/MYH6 ratio in both the epicardial and epicardial-derived cocultures, akin to the vCMEPC(+SB)_bulk (cluster 3) seen in the 2D scRNAseq (FIG. 13C). The presence of EPD-FBs ameliorated this effect and led to a slight increase in the MYH7/MYH6 ratio. Note, that all conditions did have a positive ratio further indicating a ventricular phenotype. The decrease in ratio present in the epicardial conditions was mostly due to a significant increase in the MYH6 (fetal isoform) as opposed to a decrease in the MYH7 (adult isoform) (FIG. 13E,F). The fibroblasts restored the MYH6 expression to the same level as the CM-only condition and resulted in a significant increase in the MYH7 expression.

Some key markers of electrophysiologic maturation such as RYR2, ATP2A2, KCNJ2, GJA1, and PLN were also assessed. The presence of GJA1 was significantly increased in the CM+EPC(+SB) condition possibly because the epicardial cells themselves were expressing large amounts of GJA1 as determined by scRNAseq (FIG. 13G). There were no significant differences in the expression levels of ATP2A2 and KCNJ2 detected between any of the cocultures with the CM controls (FIG. 13H-I). Additionally, epicardial cells alone did not influence the bulk RYR2 expressions but the CM Tri-culture conditions had a significant increase compared to the CM-only controls (FIG. 13J). Phosopholamban, or PLN, a regulator of SERCA2 or ATP2A2, was significantly decreased in both the epicardial cocultures but this effect was ameliorated with the addition of fibroblasts in the CM Tri culture condition (FIG. 13K). In summary, while epicardial cells spur a more fetal but electrochemically functional phenotype, the addition of EPD-FBs pushes the tissues one step further by sustaining electrochemical function and enhancing mechanical function.

What is claimed:

1. A method of producing engineered heart tissue (EHT), comprising:
providing a population of human pluripotent stem cell-derived cardiomyocytes (hiPSC-CMs);
contacting the hiPSC-CMs with epicardial cells during a first culture period; and
after the first culture period, introducing or generating within the culture one or more additional non-myocyte populations comprising epicardial-derived smooth muscle cells, endothelial cells, fibroblasts, or any combination thereof during a second culture period, wherein the non-myocyte populations are provided in a temporally staged manner that mimics embryonic-to-fetal cardiac development, thereby promoting maturation, patterning, multicellular organization, or functional performance of the engineered cardiac tissue.

2. The method of claim 1 and further comprising culturing the co-culture under conditions comprising transient inhibition of TGFβ signaling sufficient to maintain or expand an epicardial cell state during an initial stage; and subsequently reducing, removing, or discontinuing the inhibition of TGFβ signaling to permit lineage progression of the epicardial cells into one or more epicardial-derived cell types comprising fibroblasts, smooth muscle cells, or both, thereby mimicking an embryonic-to-fetal progression mimicry process in vitro.

3. The method of claim 1, wherein the additional non-myocyte population comprises fibroblasts.

4. The method of claim 1, wherein endothelial cells and epicardial-derived smooth muscle cells are introduced during the second culture period before or after fibroblasts are introduced.

5. The method of claim 1, wherein the additional non-myocyte populations are introduced sequentially rather than simultaneously.

6. The method of claim 1, wherein at least one of the additional non-myocyte populations is generated by directed differentiation of cells already present in the culture rather than by exogenous addition of fully differentiated cells.

7. The method of claim 6, wherein the fibroblasts are epicardial-derived fibroblasts that are generated in situ from epicardial cells.

8. The method of claim 1, wherein the epicardial-derived smooth muscle cells are generated in situ from epicardial cells or the endothelial cells are generated in situ from a vascular progenitor population present in the culture.

9. The method of claim 1, wherein the timing of directed differentiation is controlled by changing one or more pathway-modulating agents at prescribed time points.

10. The method of claim 9, wherein the pathway-modulating agents comprise a TGFβ pathway inhibitor.

11. An engineered heart tissue (EHT) composition, comprising:
cardiomyocytes derived from human induced pluripotent stem cells (hiPSCs); and
one or more additional cell populations derived from hiPSCs selected from:
epicardial cells, fibroblasts, endothelial cells, smooth muscle cells, and macrophages or myeloid lineage cells,
wherein the cells are arranged within 2D layer or a 3D matrix to form EHT that recapitulates developmental multicellular interactions of human myocardium.

12. The composition of claim 11 wherein the cells are arranged in the 3D matrix and the 3D matrix comprises a biopolymer comprising fibrin.

13. The composition of claim 11, wherein all cellular components of the EHT are derived from human induced pluripotent stem cells.

14. The composition of claim 11, wherein one or more of the cell populations comprise genetically encoded reporter constructs configured to report functional maturation or cellular state within the EHT.

15. The composition of claim 14, wherein one or more reporters selected from the group comprising reporters for structural cardiomyocyte maturation consisting of cTnT, a-actinin, and MYH7/MYH6; reporters for electrochemical maturation consisting of KCNJ2 and RYR2; reporters for electromechanical maturation consisting of GJA1 and NRAP, reporters for metabolic maturation consisting of PGCla and TOMM20; reporters for stress or injury markers consisting of NPPB and NPPA; reporters for cell death consisting of a caspase 3 activity sensor; reporters for fibroblast activation consisting of TE7/ACTA2; reporters for macrophage identity consisting of CD68, CD163, and MerTK, reporters for inflammatory signaling consisting of IL-6, TNFa, and IL-1b, and reporters for remodeling consisting of TGFb and IL-10.

16. A method of producing an engineered heart tissue (EHT), comprising:
assembling human pluripotent stem cell-derived cardiomyocytes (hiPSC-CMs) with one or more precursor or differentiated non-myocyte populations in a two-dimensional culture, a three-dimensional culture, a hydrogel, a fibrin gel, an engineered heart tissue, or a bioprinted construct; and
controlling the timing of appearance of epicardial cells before the appearance of fibroblasts and before or concurrent with the appearance of endothelial cells and smooth muscle cells, such that the EHT undergoes a staged developmental sequence that models embryonic-to-fetal cardiac multicellular interactions.

17. The method of claim 16, and further producing one or more additional cardiac-supporting cell types selected from endothelial cells, epicardial-derived smooth muscle cells, and fibroblasts, by either:
(i) introducing additional cardiac-supporting cell types into the culture at prescribed time points, or (ii) directing differentiation of cells present in the culture into said additional cardiac-supporting cell types at prescribed time points.

18. The method of claim 17, wherein the later stage is initiated by withdrawal or reduction of TGFβ inhibition.

19. The method of claim 17, wherein fibroblasts are produced later than endothelial cells or smooth muscle cells.
